(19) United States
(12) Patent Application Publication
(10) Pub. No.: US 2015/0182165 A1
(43) Pub. Date: Jul. 2, 2015

(54) NEUROPHYSIOLOGICAL TRAINING HEADSET

(71) Applicant: Neurotopia, Inc., Atascadero, CA (US)

(72) Inventors: Austin Miller, Atascadero, CA (US); Dale Dalke, Atascadero, CA (US); Daniel P. Dooley, Oklahoma City, OK (US)

(73) Assignee: Neurotopia, Inc., Atascadero, CA (US)

(21) Appl. No.: 14/218,560

(22) Filed: Mar. 18, 2014

Related U.S. Application Data
(63) Continuation-in-part of application No. 13/566,405, filed on Aug. 3, 2012.

(51) Int. Cl. A61B 5/00; A61B 5/0476

(52) U.S. Cl. CPC A61B 5/6803; A61B 5/0476

(57) ABSTRACT

Preferably, an embodiment of a neurophysiological training headset includes at least a plurality of sensor assemblies each sensor assembly secured by a retention web. Each of the plurality of sensor assemblies are positioned in contact adjacent at a predetermined location about a cranium of a subject. The preferred neurophysiological training headset further includes a headphone secured to the retention web by an attachment member, in which the attachment member provides a continuously active tensioning mechanism. The continuously active tensioning mechanism promotes continuous force induced contact adjacency of each of the sensor assemblies with the cranium of the subject.

### RELATED APPLICATION

This application is a continuation-in-part of U.S. patent application Ser. No. 13/566,405 filed on Aug. 3, 2012, entitled “Neurophysiological Capacitance Dry Sensor.”

### FIELD OF THE INVENTION

The present invention relates to the field of sensors. More particularly, the present invention relates to neurophysiological training headsets for use in collecting brainwave data from subjects, and most particularly to a neurophysiological training headset providing a continuously active tensioning mechanism.

### SUMMARY OF THE INVENTION

In accordance with preferred embodiments, a neurophysiological training headset includes at least a plurality of sensor assemblies each sensor assembly secured by a retention web. Each of the plurality of sensor assemblies are positioned in contact adjacent at a predetermined location about a cranium of a subject. The preferred neurophysiological training headset further includes a headphone secured to the retention web by an attachment member, in which the attachment member provides a continuously active tensioning mechanism. The continuously active tensioning mechanism promotes continuous force induced contact adjacency of each of the sensor assemblies with the cranium of the subject.

These and various other features and advantages that characterize the claimed invention will be apparent upon reading the following detailed description and upon review of the associated drawings.

### BRIEF DESCRIPTION OF THE DRAWINGS

The patent or application file contains at least one drawing executed in color. Copies of this patent with color drawing(s) will be provided by the Patent and Trademark Office upon request and payment of necessary fee.

The present invention is illustrated by way of example and not limitation in the figures of the accompanying drawings, in which like references indicate similar elements and in which:

FIG. 1 is a top plan view of an embodiment exemplary of the inventive sensor probe assembly.

FIG. 2 is a view in elevation of an embodiment exemplary a conductive pin of the inventive sensor probe assembly of FIG. 1.

FIG. 3 is a front side view in elevation of an embodiment exemplary of the inventive sensor probe assembly of FIG. 1.

FIG. 4 is a front side view in elevation of an embodiment exemplary of the inventive sensor probe assembly illustrative of a flexible, electrically conductive pin securement member and associated plurality of electrically conductive pins matted thereto, of an embodiment exemplary of the inventive sensor probe assembly of FIG. 1.

FIG. 5 is a top plan view of an alternate embodiment exemplary of the inventive sensor probe assembly.

FIG. 6 is a view in front elevation of an alternate embodiment exemplary of an electrically conductive pin of the inventive sensor probe assembly of FIG. 5.

FIG. 7 is a front side view in elevation of an alternate embodiment exemplary of the inventive sensor probe assembly of FIG. 5.

FIG. 8 is a front side view in elevation of an alternate embodiment exemplary of the inventive sensor probe assembly illustrative of a flexible, electrically conductive pin securement member and associated plurality of electrically conductive pins matted thereto, of an embodiment exemplary of the inventive sensor probe assembly of FIG. 5.

FIG. 9 is a front elevation view of an embodiment exemplary of an electrically conductive pin of FIG. 6, showing a head portion, a tip portion, and a body portion disposed there between.

FIG. 10 is a front elevation view of an embodiment exemplary of an electrically conductive pin of FIG. 2, showing a head portion having a convex shape, a tip portion, and a body portion disposed there between.

FIG. 11 is a front elevation view of an alternate embodiment exemplary of an electrically conductive pin of FIG. 2, showing a head portion having a concave shape, a tip portion, and a body portion disposed there between.

FIG. 12 is a front elevation view of an embodiment exemplary of an electrically conductive pin of FIG. 2, showing a head portion having a substantially flat top surface, a tip portion, and a body portion disposed there between.

FIG. 13 is a partial cutaway front elevation view of an alternate tip configuration for any of the electrically conductive pins of FIG. 9, 10, 11, or 12.

FIG. 14 is a cross-section, partial cutaway front elevation view of an alternate tip configuration for any of the electrically conductive pins of FIG. 9, 10, 11, or 12.

FIG. 15 is a partial cutaway front elevation view of an alternative tip configuration for any of the electrically conductive pins of FIG. 9, 10, 11, or 12.

FIG. 16 is a partial cutaway front elevation view of an alternate tip configuration for any of the electrically conductive pins of FIG. 9, 10, 11, or 12.

FIG. 17 is a flowchart of a method of producing an embodiment exemplary of the inventive sensor probe assembly of either FIG. 1 or FIG. 5.

FIG. 18 is a front elevation view in cross section of an embodiment exemplary of the present novel sensor assembly.

FIG. 19 is a bottom plan view of the novel sensor assembly of FIG. 18.

FIG. 20 is a front elevation view exploded view in cross section of the novel sensor assembly of FIG. 18.

FIG. 21 is a front elevation view in cross section of an alternate embodiment exemplary of the present novel sensor assembly.

FIG. 22 is a side elevation view in cross section of the alternate embodiment exemplary of the present novel sensor assembly of FIG. 21.

FIG. 23 is a side elevation view in cross section of the alternate embodiment exemplary of the present novel sensor assembly of FIG. 21, communicating with a brainwave processing system.

FIG. 24 is a schematic of a preferred signal processing circuit of the embodiment exemplary of the present novel sensor assembly of either FIG. 18, 21, or 23.

FIG. 25 is a flowchart of a method of using an embodiment exemplary of the inventive sensor assembly of either FIG. 18, 21, or 23.

FIG. 26 is a cross section exploded view in elevation of an embodiment of a novel capacitance probe sensor assembly of the present invention.

FIG. 27 is a cross section view in elevation of the embodiment of the novel capacitance probe sensor assembly of FIG. 26.

FIG. 28 is a cross section partial exploded view in elevation of an alternate embodiment of a novel capacitance probe sensor assembly of the present invention.

FIG. 29 is a cross section view in elevation of the embodiment of the novel capacitance probe sensor assembly of FIG. 28.

FIG. 30 is a partial exploded view of an alternative embodiment of a novel capacitance probe of the present invention.

FIG. 31 is a further cross section, partial exploded view in elevation of an alternative embodiment of a novel capacitance probe sensor assembly of the present invention of FIG. 30.

FIG. 32 is a cross section view in elevation of the embodiment of the novel capacitance probe sensor assembly of FIG. 31.

FIG. 33 is a cross section, partial exploded view of an alternate alternative embodiment of a novel capacitance probe sensor assembly of the present invention.

FIG. 34 is a cross section view in elevation of the embodiment of the novel capacitance probe sensor assembly of FIG. 33.

FIG. 35 is a cross section exploded front elevation view of a different alternate embodiment of the present capacitance probe sensor assembly invention.

FIG. 36 is a partial cutaway, cross section, front elevation view of the different alternate embodiment of the present capacitance probe sensor assembly invention of FIG. 38.

FIG. 37 is a side elevation view of a unique embodiment of a dry sensor system, which accommodates resistive as well as capacitance sensing probes and includes a retention web and supports a set of headphones.

FIG. 38 is a view in elevation of an embodiment exemplary of novel neurophysiological training headset.

FIG. 39 is a view in elevation of a frame assembly of a retention web of the neurophysiological training headset of FIG. 38.

FIG. 40 is a side view in elevation of the neurophysiological training headset of FIG. 38.

FIG. 41 is a side view in elevation of a tension housing of the neurophysiological training headset of FIG. 40.

FIG. 42 is a top plan view of a cover plate of the tension housing of the neurophysiological training headset of FIG. 40.

FIG. 43 is a bottom plan view of an access cover of the tension housing of the neurophysiological training headset of FIG. 38.

FIG. 44 is a top plan view of a shape retention member of an attachment member of the neurophysiological training headset of FIG. 40.

FIG. 45 is a side perspective view of the shape retention member of FIG. 44, nested in a shape retention channel of the frame assembly of FIG. 39.

FIG. 46 is a side perspective view of a mounting flange of the frame assembly of FIG. 39.

FIG. 47 is a side view a guide plate of the tension housing of FIG. 41

FIG. 48 is a side view of a slide plate of a slide structure of FIG. 40.

FIG. 49 is a top plan view of the neurophysiological training headset of FIG. 40.

FIG. 50 is a top plan view of a sensor of the plurality of sensors of the neurophysiological training headset of FIG. 40.

FIG. 51 is an alternate top plan view of a sensor of the plurality of sensors of the neurophysiological training headset of FIG. 40.

FIG. 52 is an additional alternate top plan view of a sensor of the plurality of sensors of the neurophysiological training headset of FIG. 40.

FIG. 53 is a top plan view of a mounting aperture for a sensor of the plurality of sensors of the neurophysiological training headset of FIG. 40.

FIG. 54 is a top plan view of a sensor securement cap for one of the plurality of sensors of the neurophysiological training headset of FIG. 40.

FIG. 55 is a top plan view of a pliable compliant member, which cooperates with sensor securement cap of FIG. 55 to provide six degrees of freedom for each of the plurality of sensors of the neurophysiological training headset of FIG. 40.

FIG. 56 shows a functional block diagram of a neurophysiological training system, constructed in accordance with various embodiments disclosed and claimed herein.

### DESCRIPTION OF PREFERRED EMBODIMENTS

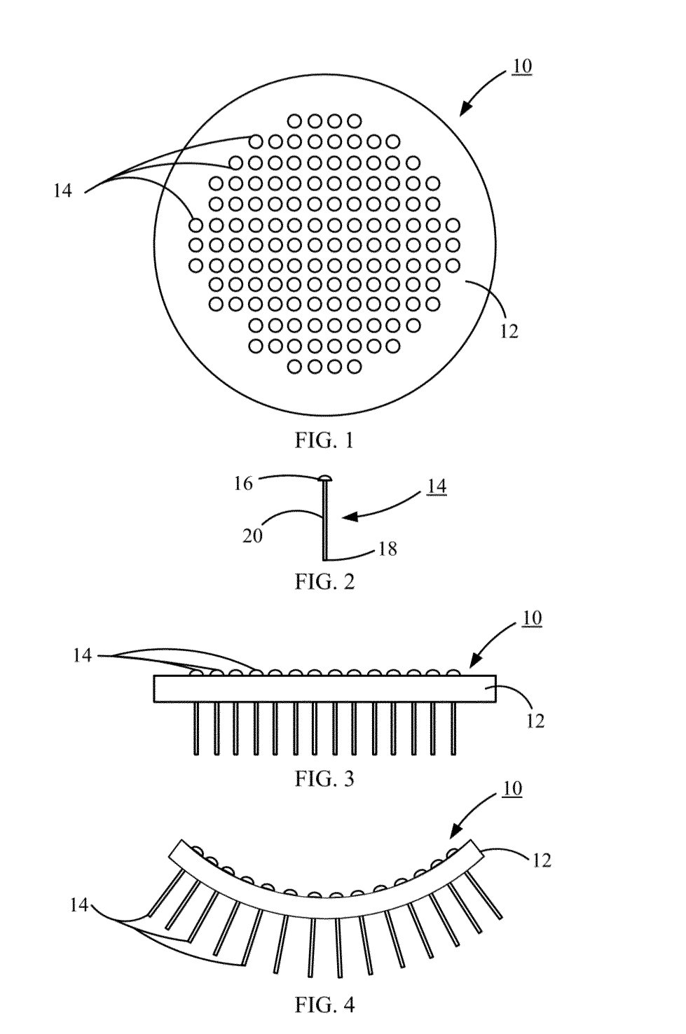

It will be readily understood that elements of the present invention, as generally described and illustrated in the figures herein, could be arranged and designed in a wide variety of different configurations. Referring now in detail to the drawings of the preferred embodiments, a sensor probe assembly 10, of FIG. 1, (also referred to herein as assembly 10) of a first preferred embodiment, while useable for a wide variety of bio-physiological sensing applications, it is particularly adapted for use as neurophysiological signal sensor component. Accordingly, the assembly 10 of the first preferred embodiment, of FIG. 1, will be described in conjunction with the merits of the use of the sensor probe assembly 10 as a neurophysiological signal sensor component.

In a preferred embodiment of FIG. 1, the sensor probe assembly 10 includes at least a conductive pin securement member 12, which hosts a plurality of conductive pins 14. Preferably, the plurality of conductive pins 14 are electrically conductive, and when in pressing contact with the conductive pin securement member 12, as shown by FIG. 3, form the sensor probe assembly 10 that yields a low impedance neurophysiological signal sensor component.

In a preferred embodiment, the conductive pins 14, an example of which is shown by FIG. 2, include at least a head portion 16, a tip portion 18, and a body portion 20 disposed between the head portion 16 and the tip portion 18. Preferably, each conductive pin 14 is formed from a non-corrosive material, such as stainless steel, titanium, bronze, or a gold plating on a rigid substrate selected from a group including at least polymers and metals. Preferably, the head portion 16 has a diameter greater than the diameter of the body portion 20.

As shown by FIG. 4, the conductive pin securement member 12 is preferably flexible and formed from a polymer. The electrical conductivity of the conductive pin securement member 12 is preferably attained by the inclusion of conductive particles embedded within the polymer. One such combination is a carbon filed silicon sheet material provided by Stockwell Elastomerics, Inc. of Philadelphia, Pa. However, as known in the art, conductive polymers may be formed from a plurality of polymer materials filled with conductive particles, the shape of which may be formed using well known manufacturing techniques that include at least molding, extrusion dies and sliced to thickness, formed in sheets and: die cut; cut with hot wire equipment; high pressure water jets, or steel rule dies.

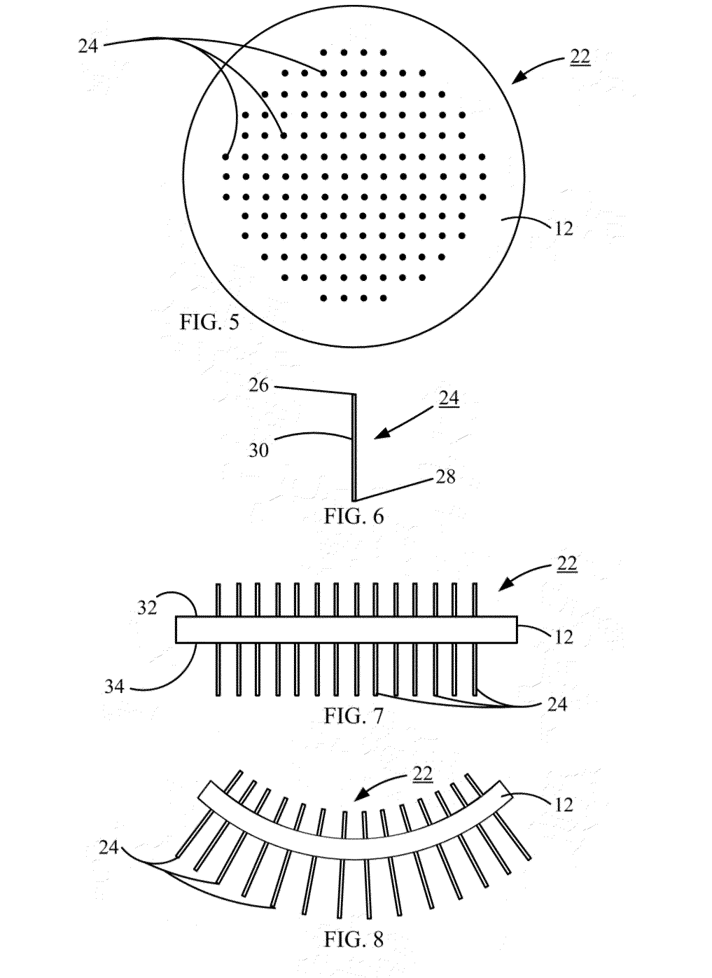

FIG. 5 shows an alternate embodiment of a sensor probe assembly 22, which is preferably formed from the conductive pin securement member 12, and a plurality of alternate preferred conductive pins 24. As shown by FIG. 6, preferably each alternate preferred conductive pin 24 includes a head portion 26, a tip portion 28, and a body portion 30, wherein the head portion 26 and the tip portion 28 have diameters substantially equal to the body portion 30. However, a skilled artisan will appreciate that conductive pins may have head, tip and body portion diameters different from one another. For example, the body portion may have a diameter greater than either the tip portion or head portion to accommodate insert molding of the conductive pins into a conductive pin securement member. It is further understood that the conductive pins may take on a profile that includes a bend in the body, tip, or head portions, as opposed to the cylindrical configuration of any suitable cross section geometric shape of the conductive pins shown by FIG. 2 and FIG. 6. It is still further understood, that the conductive pins may be formed by a plurality of individual components, including without limitation a spring, or may be formed from a coiled or other form of spring alone.

As with the preferred conductive pins 14, the alternate preferred conductive pins 24 are formed from a non-corrosive material, such as stainless steel, titanium, bronze, or a precious metal plating on a rigid substrate selected from a group including at least polymers and metals.

FIG. 7 shows the conductive pins 24 protruding through each the top and bottom surfaces, 32 and 34 respectfully, to accommodate improved conductivity of the alternate sensor probe assembly 22, with mating components. While FIG. 8 shows that the alternate sensor probe assembly 22 preferably retains the flexibility characteristics of sensor probe assembly 10 of FIG. 4.

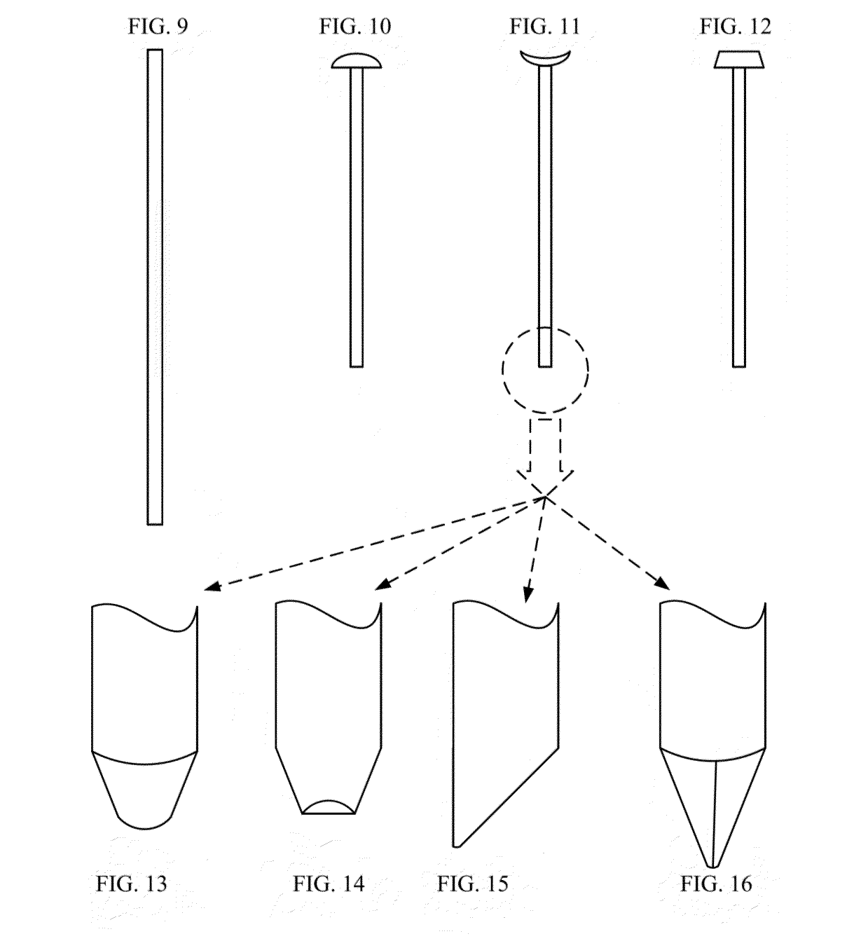

FIGS. 9, 10, 11, and 12 show just a few of a plurality of head configurations suitable for use on conductive pins. The particular configuration selected is a function of the device or component with which the conductive pins electrically cooperate. When a connector is used to interface with the sensor probe assembly, such as 10 or 22, the precise configuration will depend on the type and configuration of the pins associated with the connector, including whether the pins are male or female pins.

FIGS. 13, 14 (a cross section view), 15, and 16 show just a few of a plurality of tip configurations suitable for use on conductive pins. The particular configuration selected is a function of the materials used to form the conductive pins, and the environment in which the conductive pin will be placed. Examples of the use environment include where on the cranium the sensor will be placed, whether hair is present, and the sensitivity of the subject to the tips of the conductive pins.

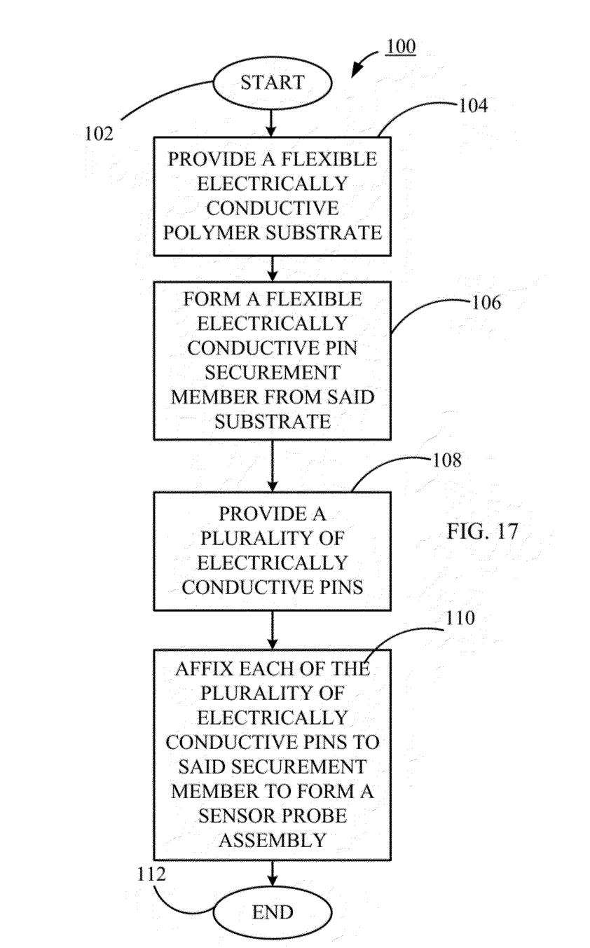

FIG. 17 shows a method 100, of making a sensor probe assembly, such as 10 or 22. The method begins at start step 102, and proceeds to process step 104, where a flexible conductive pin securement material is provided (also referred to herein as a flexible, electrically conductive, polymer substrate). At process step 106, a flexible, electrically conductive, pin securement member (such as 12) is formed from the flexible, electrically conductive, polymer substrate.

The process continues at process step 108, a plurality of electrically conductive pins (such as 14) is provided. At process step 110, each of the plurality of electrically conductive pins are affixed to the flexible, electrically conductive, pin securement member, and the process concludes at end process step 112 with the formation of a sensor probe assembly.

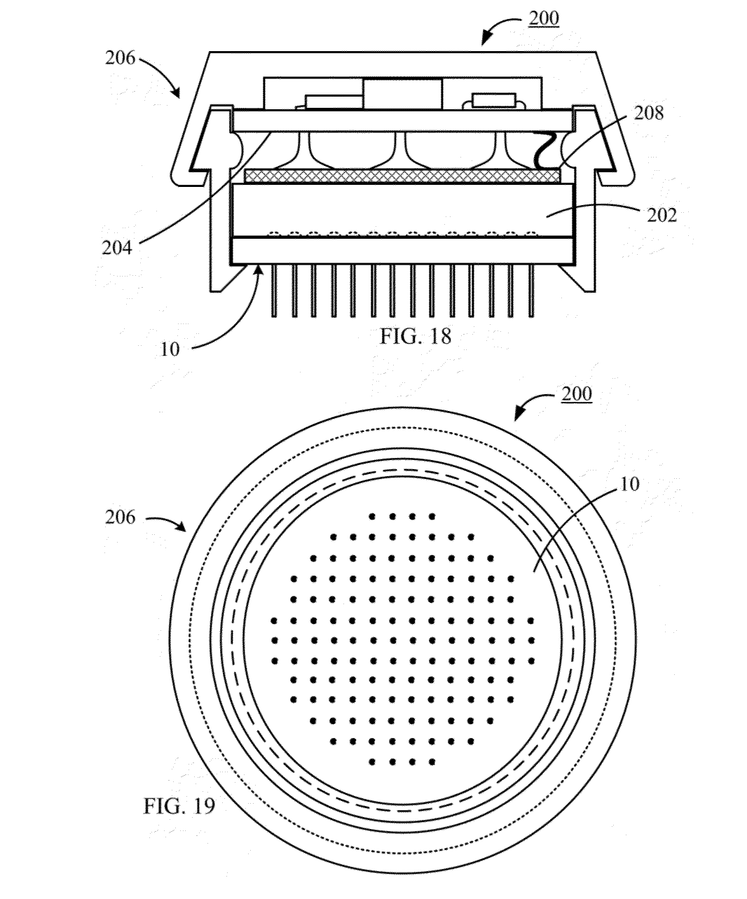

Turning to FIG. 18, shown therein is an embodiment of a novel, inventive, sensor assembly 200. Preferably, the sensor assembly 200 includes at least a sensor probe assembly 10, which provides a plurality of conductive pins 14, and a compressible electrically conductive member 202, in electrical communication with the sensor probe assembly. Preferably, the compressible electrically conductive member 202 is formed from a polyurethane polymer filled with conductive particles, which are preferably carbon particles. One such combination is a low density black conductive Polyurethane open cell flexible conductive foam material provided by Correct Products, Inc. of Richardson, Tex. However, as known in the art, conductive polymers may be formed from a plurality of polymer materials filled with conductive particles, the shape of which may be formed using well known manufacturing techniques that include at least molding, extrusion dies and sliced to thickness, formed in sheets and: die cut; cut with hot wire equipment; high pressure water jets, or steel rule dies.

As further shown by FIG. 18, the embodiment of the novel, inventive, sensor assembly 200 includes at least a signal processing circuit 204, in electrical communication with the compressible electrically conductive member 202, and a housing 206, confining the sensor probe assembly 10, the compressible electrically conductive member 202, and the signal processing circuit 204, to form the sensor assembly 200.

FIG. 19 shows the preferred embodiment of the sensor assembly 200 to be of a continuous curvilinear configuration, however, those skilled in the arts will recognize that any geometric shape may be presented by the sensor assembly 200. It is further noted that the sensor probe assembly 10, is confined by the housing 206 in such a manner that the sensor probe assembly 10, can be replaced without the disassembly of the entire sensor assembly 200.

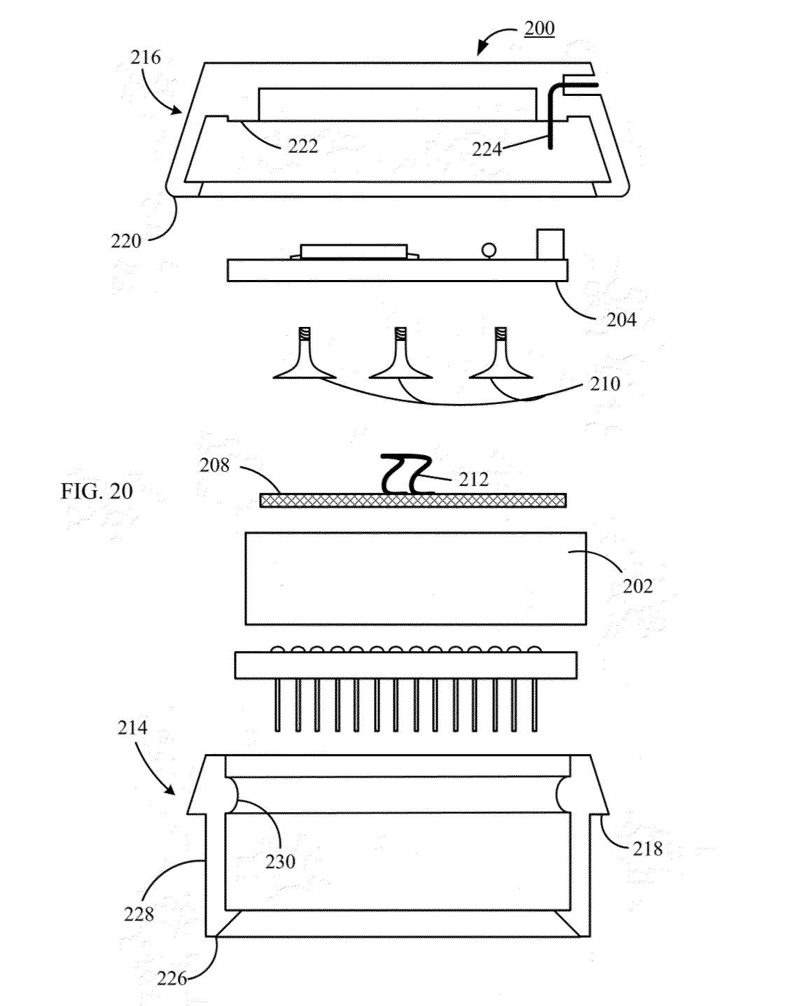

The right side cross-section view and elevation of the preferred embodiment of the sensor assembly 200 of FIG. 20, reveals a rigid conductive member 208, and a plurality of standoffs 210, disposed between the signal processing circuit 204, and the electrically conductive member 202 (shown in its decompressed form). Preferably, the rigid conductive member 208 is in electrical interaction with a signal conductor 212, and the signal conductor 212 is in electrical communication with signal processing circuit 204. These standoffs 210, are preferably attached to the signal processing circuit 204, and function to provide a slight compressive load on the compressible electrically conductive member 202. The compressive load allows for decompression of the compressible electrically conductive member 202 while the probe assembly is being exchanged. This particular feature promotes stability of the rest of components within the housing 206, when the sensor probe assembly is absent from the remaining components of the sensor assembly 200.

As is further shown by FIG. 20, the housing 206, of FIG. 18, preferably includes a component chamber 214, and a confinement cover 216. The component chamber 214 preferably includes a confinement cover retention feature 218, which interacts with a retention member 220 of the confinement cover 216. In a preferred embodiment, the confinement cover 216 “snaps” onto the component chamber 214. In a preferred embodiment, the component chamber 214 and the confinement cover 216 are formed from a shape retaining material that provides sufficient flexibility to allow the retention member 220 of the confinement cover 216 to pass by the confinement cover retention feature 218 of the component chamber 214, and then lock together the confinement cover 216 with the component chamber 214. As those skilled in the art will recognize that there are a number of engineering materials suitable for this purpose including, but not limited to, metals, polymers, carbon fiber materials, and laminates.

In the preferred embodiment of the sensor assembly 200, the confinement cover 216 further includes at least a signal processing circuit retention feature 222 and a connector pin 224 supported by the signal processing circuit retention feature 222, while the component chamber 214 further includes at least: a sensor probe assembly retention feature 226; a side wall 228 disposed between the confinement cover retention feature 218 and the sensor probe assembly retention feature 226: and a holding feature 230 provided by the side wall 228 and adjacent in the confinement cover retention feature 218.

In the preferred embodiment of the sensor assembly 200, the compressibility of the compressible electrically conductive member 202 promotes an ability to change out the sensor probe assembly 10, without disturbing the interaction of the signal processing circuit 204 and the rigid conductive member 208, or to change out the processing circuit 204 and the rigid conductive member 208 without disturbing the sensor probe assembly 10. When the sensor probe assembly 10 is removed from the preferred embodiment of the sensor assembly 200, the compressible electrically conductive member 202 explains to interact with the sensor probe assembly retention feature 226 thus maintaining the rigid conductive number 208 in pressing contact with standoffs 210. When the signal processing circuit 204, standoffs 210, and the rigid conductive member 208 are removed from the preferred embodiment of the sensor assembly 200, the compressible electrically conductive member 202 explains to interact with the holding feature 230 to preclude the inadvertent removal of the sensor probe assembly 10 from communication with the sensor probes assembly retention feature 226.

As will be recognized by skilled artisans, it is the collaborative effect of the pin or pins 14 of the sensor probe assembly 10 interacting with the cranium of the subject that promotes transference of brainwave signals of the subject to the signal processing circuit 204. To promote the conveyance of the brainwave signal, the sensor probe assembly 10 further provides a conductive pin securement member 12 cooperating in retention contact with the plurality of conductive pins 14.

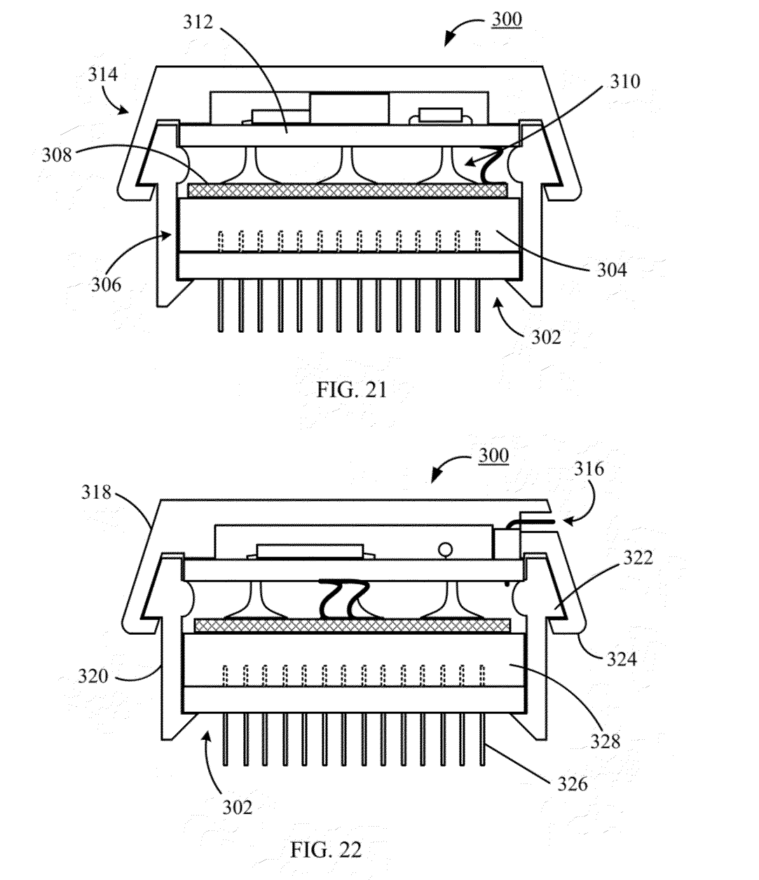

FIG. 21 shows an alternate preferred embodiment of a novel, inventive, standalone sensor assembly 300. Preferably, the standalone sensor assembly 300 includes at least an electrically conductive member 302 forming a first plate 304 of a capacitor 306, a dielectric material 308, adjacent the first plate 304, a second plate 310 of the capacitor 306 communicating with the dielectric material 308, and a signal processing circuit 312 in electrical communication with said dielectric material 308. FIG. 21 further shows a housing 314 confining the first plate 304 of the capacitor 306, the dielectric material 308, the second plate 310, and the signal processing circuit 312 to form the standalone sensor assembly 300.

FIG. 22 shows the standalone sensor assembly 300 further includes a communication port 316, useful for transferring processed signals to an external system for analysis, and that the housing 314 preferably includes a component chamber 318, and a confinement cover 320. The component chamber 318 preferably includes a confinement cover retention feature 322, which interacts with a retention member 324 of the confinement cover 320. In a preferred embodiment, the confinement cover 320 “snaps” onto the component chamber 318.

In a preferred embodiment, the component chamber 318 and the confinement cover 320 are formed from a shape retaining material that provides sufficient flexibility to allow the retention member 324 of the confinement cover 320 to pass by the confinement cover retention feature 322 of the component chamber 318, and then lock together the confinement cover 320 with the component chamber 318. As those skilled in the art will recognize that there are a number of engineering materials suitable for this purpose including, but not limited to, metals, polymers, carbon fiber materials, and laminates.

In a preferred embodiment, the electrically conductive member 302 forming the first plate 304 of the capacitor 306 includes at least, but is not limited to, a plurality of at least partially insulated pins 326, communicating with a conductive member 328, wherein the conductive member is in direct contact adjacency with the dielectric material 308. In operation, the voltage potential is present between the first plate 304 and the second plate 310, which results in a charge build up, and it is the level of the charge build up that is processed by the signal processing circuit 312. The plurality of at least partially insulated pins 326, each preferably have four degrees of freedom i.e.: yaw; pitch; roll; and z axis. The multiple degrees of freedom accommodates the topography differences in the cranium of different subjects, to promote a subject adaptable, alternate preferred embodiment of the novel, inventive, standalone sensor assembly 300.

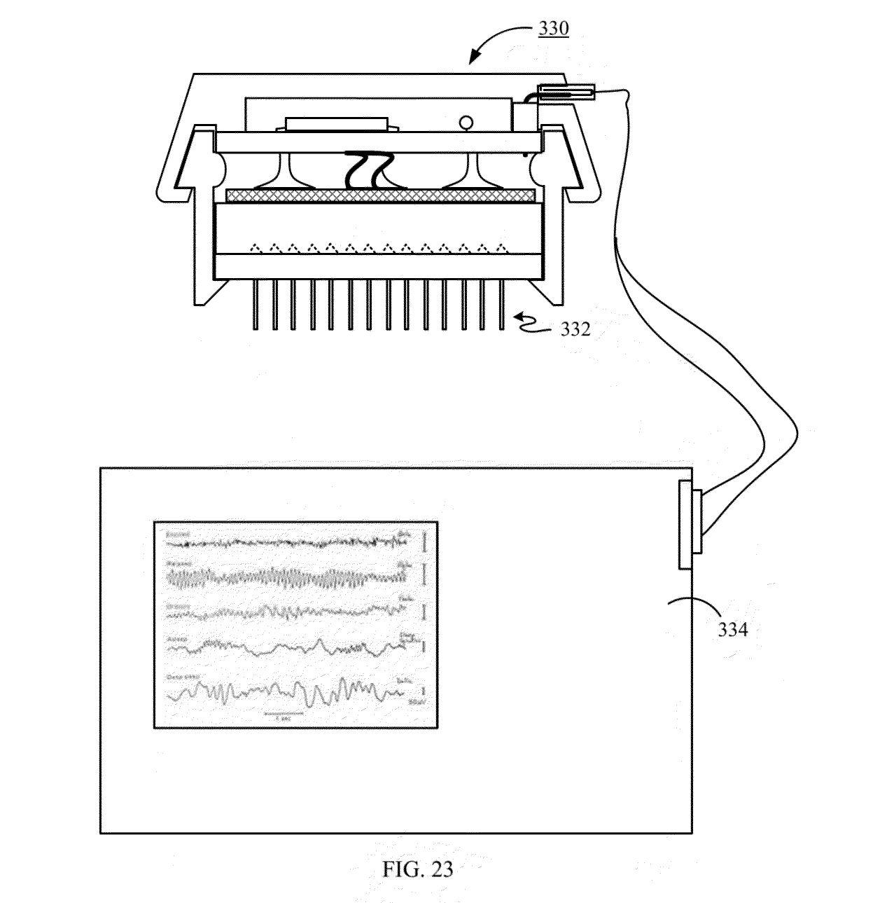

FIG. 23 shows an alternative preferred embodiment of the novel, inventive, standalone sensor assembly 330, having a plurality of alternate conductive pins 332; however, the remaining components are substantially equal to the corresponding remaining components of the preferred embodiment of the novel, inventive, standalone sensor assembly 200. Further shown by FIG. 23, is a brainwave processing system 334, which may be, for example, an Electroencephalography (EEG) 334.

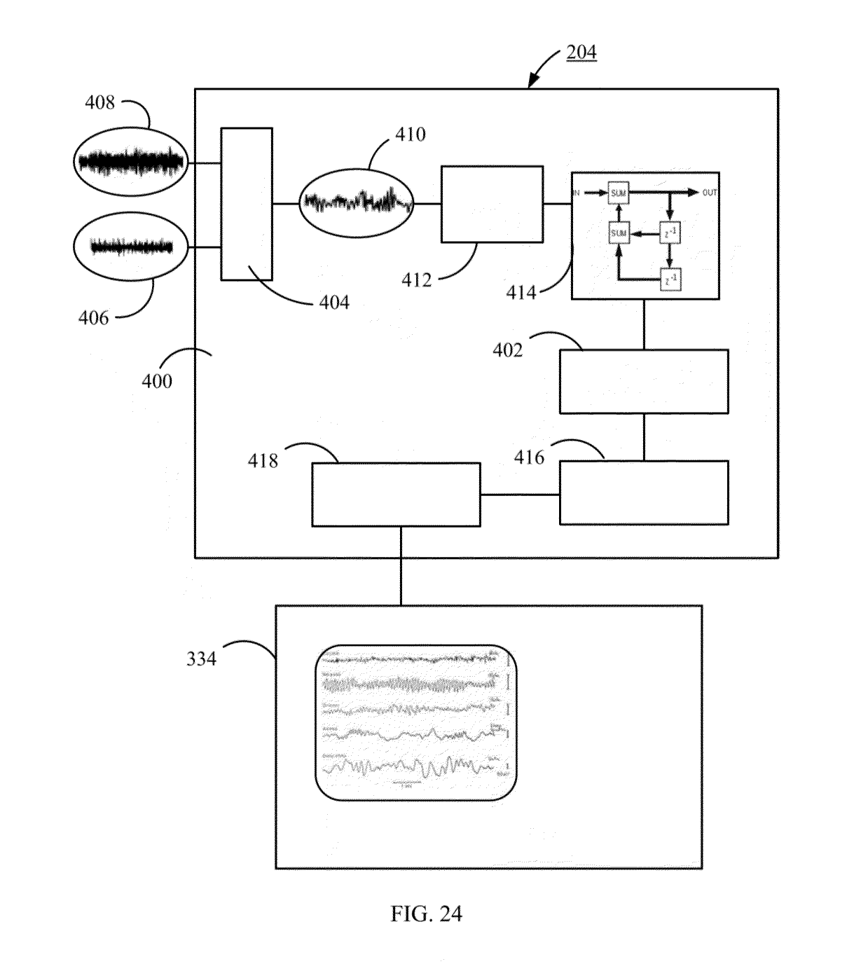

As is shown by FIG. 24, a preferred embodiment of the signal processing circuit 204 includes at least, but is not limited to, a printed circuit member 400, and a processor 402, interacting with said printed circuit member 400, the processor receiving signals from a sensor probe assembly, such as 200 of FIG. 18, and communicating the signals to a brainwave processing system, such as 334 of FIG. 23.

The preferred embodiment of the signal processing circuit 204 further includes at least, but is not limited to, a differential amplifier 404, interacting with the printed circuit member 400, a reference signal 406 communicating with the differential amplifier 404, and a subject signal 408 provided by a sensor probe assembly, such as 200 of FIG. 18, when the sensor probe assembly 200 is in electrical contact with a cranium of a subject. Preferably, the differential amplifier 404 compares the reference signal 406 to the subject signal 408 and discards common signal patterns presented by said reference and subject signals, 404 and 406, to provide a native brainwave signal 410, of the subject.

Further, the preferred embodiment of the signal processing circuit 204 includes at least, but is not limited to, an analog to digital converter with a digital signal processing core 412, interacting with the differential amplifier 404 and processing the native brainwave signal 410, provided by the differential amplifier 404, and outputting a digital signal representative of the native brainwave signal, and an infinite impulse response filter 414, interacting with the analog to digital converter 412, to serve as a band pass filter for said digital signal.

Still further, the preferred embodiment of the signal processing circuit 204 shown in FIG. 24, includes at least, but is not limited to, a memory 416, also referred to herein as a buffer 416, communicating with the processor 402, and storing processed native brainwave signals, and a communication port 418 communicating with the buffer 416, the communication port is preferably responsive to the processor 402 for communicating processed native brainwave signals to the brainwave processing system 334.

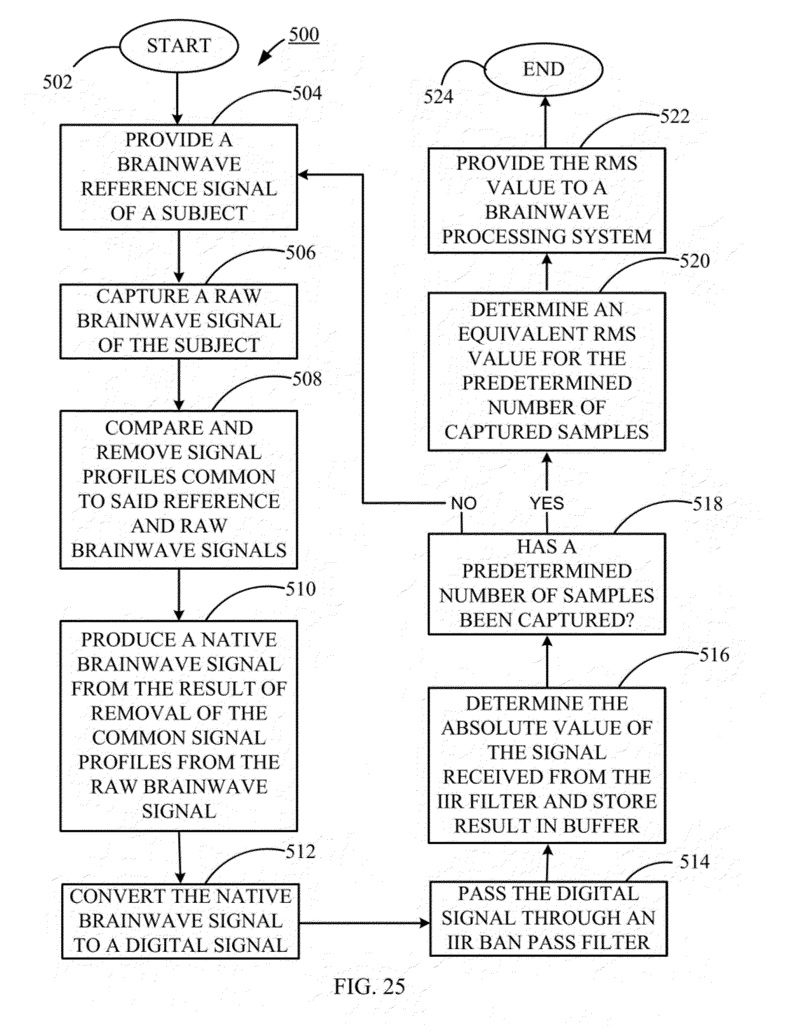

FIG. 25 shows a method 500, of using a signal processing circuit, such as 400, of FIG. 24. The method begins at start step 502, and proceeds to process step 504, where a brainwave reference signal (such as 406) of a subject is provided. At process step 506, a raw brainwave signal (such as 408) of the subject is captured. At process step 508, the signal profiles of the reference and raw brainwave signals are compared, and signal profiles common to both are removed, and at process step 510, a native brainwave signal (such as 410) is produced from the result of the removal of signal profiles common to both the reference and raw brainwave signals.

The process continues at process step 512, where the native brainwave signal is converted to a digital band of frequency signal, and passed to an HR ban pass filter (such as 414) at process step 514. At process step 516, an absolute value of the digitized signal received from the IRR filter is determined by a processor (such as 402). It is noted that in a preferred embodiment the IIR filter is programmable and responsive to the processor, and that multiple IIR filters may be employed to capture a multitude of discrete ban frequencies (typically having about a 5 Hz spread, such as 10 to 15 Hz out of a signal having a frequency range of about 0.5 Hz to 45 Hz)), or the programmable IIR filter may be programmed to collect a certain number of discrete, common frequency band samples, each sample obtained over a predetermined amount of time, and then reprogrammed to obtain a number of different, discrete, common frequency band samples.

The process continues at process step 518, where the processor determines if a predetermined number of samples of the absolute value each discrete band frequency of interest has been stored in a buffer (such as 416). If the number of captured desired samples has not been met, the process reverts to process step 504. If the number of captured desired samples has been met, the process proceeds to process step 520. At process step 520, the processor determines an equivalent RMS (root mean square) value for each of the plurality of discrete band frequency, absolute value sets of samples, and those values are provided to a brainwave processing system (such as 334) at process step 522. At process step 524, the process ends.

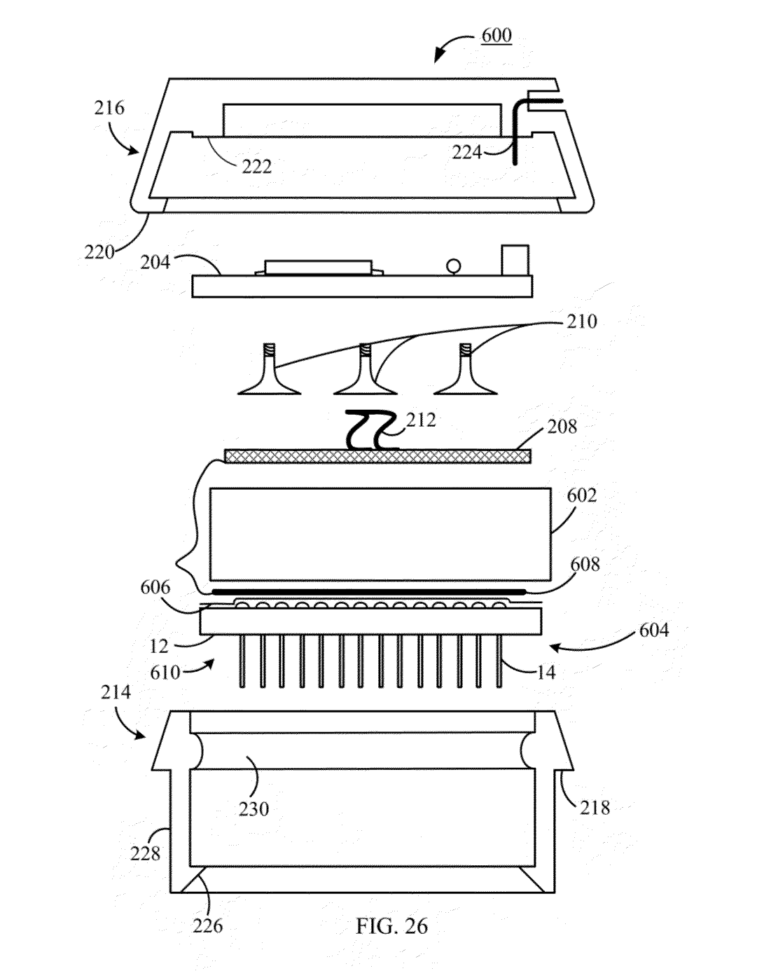

The right side cross-section view and elevation of the preferred embodiment of the sensor assembly 600 of FIG. 26, reveals a rigid conductive member 208, and a plurality of standoffs 210, disposed between the signal processing circuit 204, and a compressible compliance member 602 (shown in its decompressed form). Preferably, the rigid conductive member 208 is in electrical interaction with a signal conductor 212, and the signal conductor 212 is in electrical communication with the signal processing circuit 204. These standoffs 210, are preferably attached to the signal processing circuit 204, and function to provide a slight compressive load on the compressible compliance member 602. The compressive load allows for decompression of the compressible compliance member 602 while a sensor probe assembly 604 is being exchanged. This particular feature promotes stability of the rest of components within the housing 206, when the sensor probe assembly is absent from the remaining components of the sensor assembly 600.

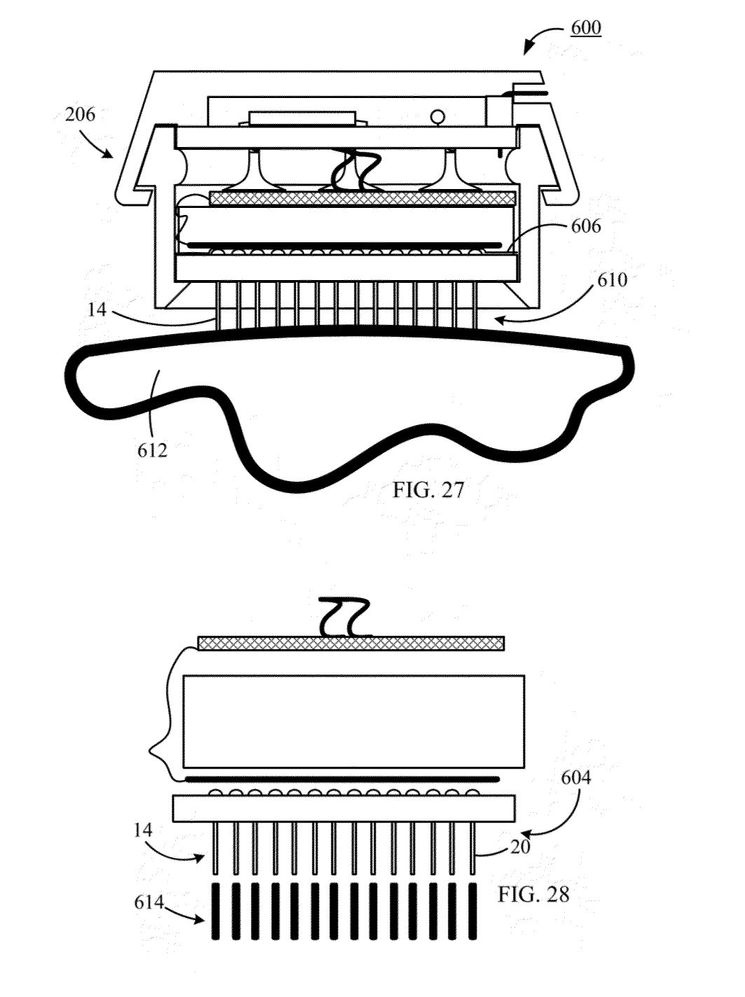

As is further shown by FIG. 26, the housing 206, of FIG. 27, preferably includes the component chamber 214, and the confinement cover 216. The component chamber 214 preferably includes the confinement cover retention feature 218, which interacts with the retention member 220 of the confinement cover 216. In a preferred embodiment, the confinement cover 216 “snaps” onto the component chamber 214. In a preferred embodiment, the component chamber 214 and the confinement cover 216 are formed from a shape retaining material that provides sufficient flexibility to allow the retention member 220 of the confinement cover 216 to pass by the confinement cover retention feature 218 of the component chamber 214, and then lock together the confinement cover 216 with the component chamber 214. As those skilled in the art will recognize that there are a number of engineering materials suitable for this purpose including, but not limited to, metals, polymers, carbon fiber materials, and laminates.

In the preferred embodiment of the sensor assembly 600, the confinement cover 216 further includes at least the signal processing circuit retention feature 222 and the connector pin 224 supported by the signal processing circuit retention feature 222, while the component chamber 214 further includes at least: the sensor probe assembly retention feature 226; the side wall 228 disposed between the confinement cover retention feature 218 and the sensor probe assembly retention feature 226; and the holding feature 230 provided by the side wall 228 and adjacent the confinement cover retention feature 218.

In the preferred embodiment of the sensor assembly 600, the compressibility of the compressible compliance member 602 promotes an ability to change out the sensor probe assembly 604, without disturbing the interaction of the signal processing circuit 204 and the rigid conductive member 208, or to change out the processing circuit 204 and the rigid conductive member 208 without disturbing the sensor probe assembly 10. When the sensor probe assembly 604 is removed from the preferred embodiment of the sensor assembly 600, the compressible compliance member 602 explains to interact with the sensor probe assembly retention feature 226 thus maintaining the rigid conductive number 208 in pressing contact with standoffs 210. When the signal processing circuit 204, standoffs 210, and the rigid conductive member 208 are removed from the preferred embodiment of the sensor assembly 600, the compressible compliance member 602 explains to interact with the holding feature 230 to preclude the inadvertent removal of the sensor probe assembly 604 from communication with the sensor probes assembly retention feature 226.

In a preferred embodiment, the sensor probe 604 is a capacitance sensor probe formed by at least a first conductive member, which in the present embodiment is a plurality of conductive pins 14; a conductive pin securement member 12 cooperating in retention contact with the plurality of conductive pins 14; a dielectric material 606 in pressing contact with the plurality of conductive pins 14; and a second conductive member, which in the present embodiment is a metallic foil 608, in pressing contact with the dielectric material 606. The plurality of conductive pins 14, form a first plate of a capacitor 610, while the conductive foil 608 forms a second plate of capacitor 610.

FIG. 27 shows that the sensor probe assembly 610, with the first conductive member (the plurality of conductive pins 14) of the capacitance sensor probe 604, is in electrical contact with a cranium 612 (shown in partial cutaway) of a subject. In this embodiment configuration, the brain waves of the subject are conducted by the conductive pins 14 to the dielectric material 606.

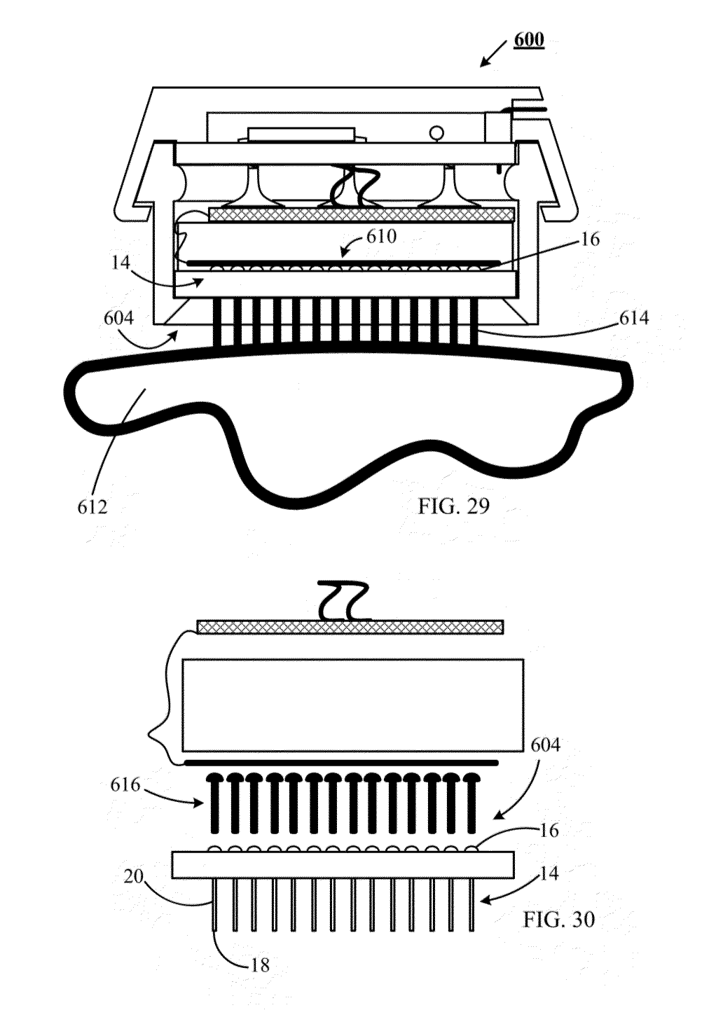

FIG. 28 shows an alternate configuration of the capacitance sensor probe 604, which features dielectric material 614, which preferably coats the body portion 20 of each of the plurality of conductive pins 14 coating. As shown by FIG. 29, in this embodiment configuration of the sensor assembly 600, the dielectric material 614 is in electrical contact with the cranium 612 of the subject, making the cranium 612 the second plate of the capacitance sensor probe 604, and the collective heads 16 of the plurality of conductive pins 14 the first plate of the capacitor 610.

FIG. 29 shows that the sensor assembly 600 with the first conductive member (the plurality of conductive pins 14) of the capacitance sensor probe 604 is in electrical contact with a cranium 612 (shown in partial cutaway) of a subject. In this embodiment configuration, the brain waves of the subject are conducted by the conductive pins 14 to the dielectric material 606.

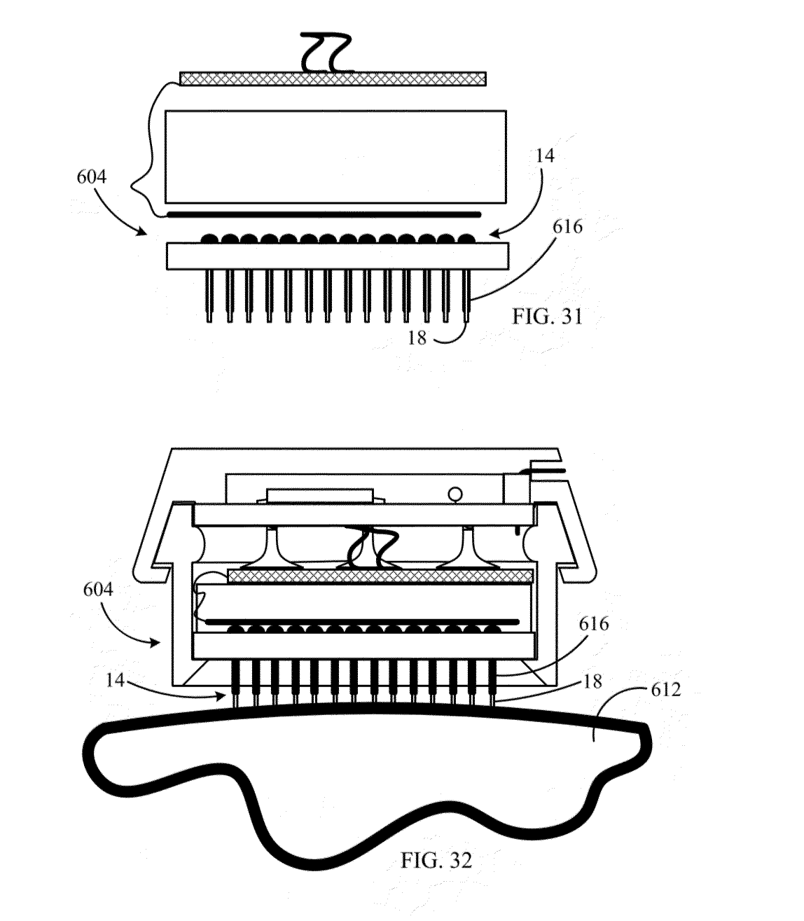

FIG. 30 shows an alternate configuration of the capacitance sensor probe 604, which features dielectric material 616, which preferably coats the head portion 16, and the body portion 20 of each of the plurality of conductive pins 14 coating, leaving the tip portion 18 uncoated, as shown by FIG. 31. In the embodiment configuration of the capacitance sensor probe 604 shown by FIG. 32, the dielectric material 616 is in electrical contact with the metallic foil 608, making the combination of the cranium 612 and the tips 18 of the plurality of conductive pins 14 the second plate of the capacitance sensor probe 604, and the metallic foil 608 the first plate of the capacitor 610.

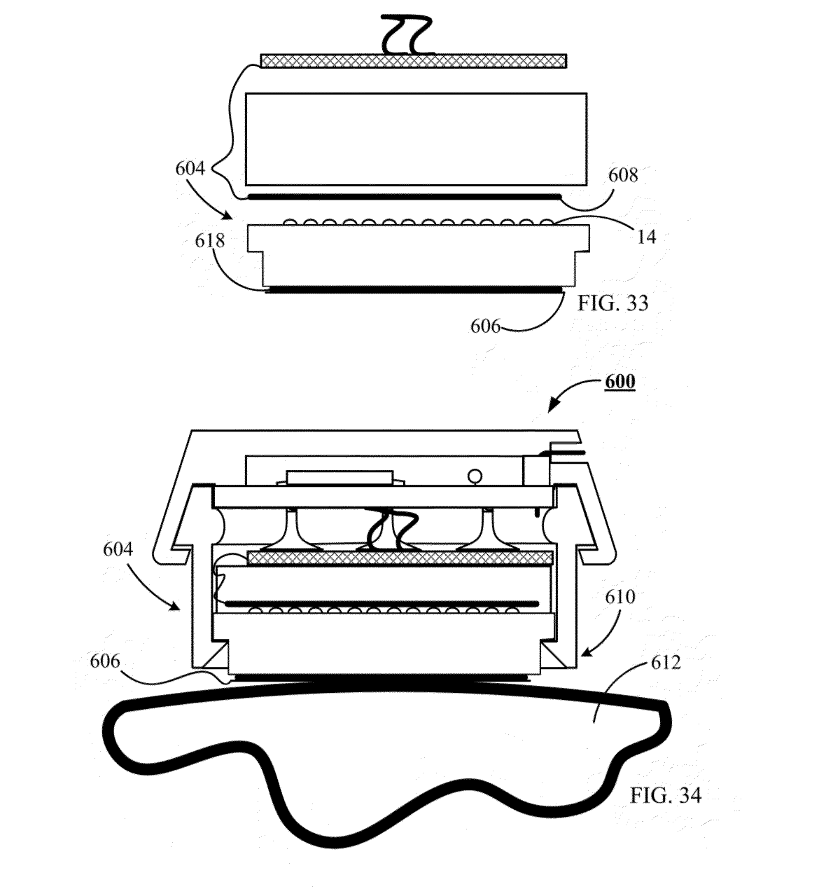

In the embodiment of the sensor assembly 600 shown by FIGS. 33 and 34, a second metallic foil 618 in combination with conductive pins 14 and the metallic foil 608 forms the first plate of the capacitor 610, the cranium 612 of the subject forms the second plate of the capacitor 610, with the dielectric material 606 disposed between the first and second plates of the capacitor 610. Accordingly, the capacitor 610 is formed when the sensor assembly 600 is held in pressing contact against the cranium 612 of the subject.

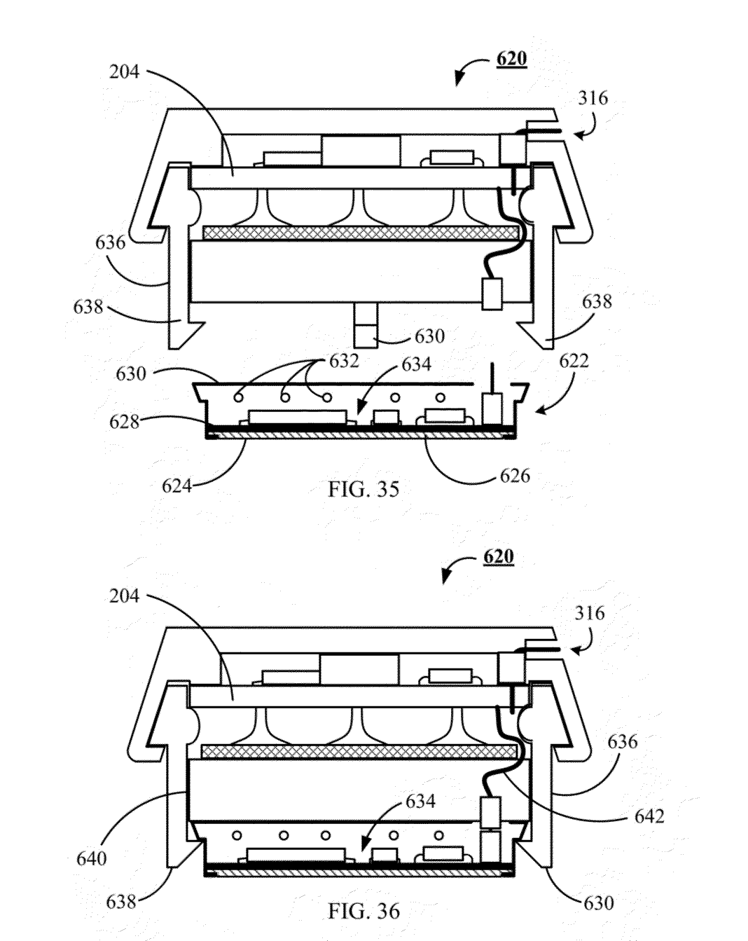

FIGS. 35 and 36 provide an alternate alternative preferred embodiment of a capacitance sensor assembly 620, which includes a capacitance probe assembly 622, communicating with the signal processing circuit 204. Preferably, the capacitance probe assembly 622 includes a first conductor 624 in direct electrical contact with a dielectric material 626, and a second conductor 628 in direct electrical contact with the dielectric material 626. The capacitance probe assembly 622 further preferably includes a capacitance probe shield 630, which provides a plurality of vent apertures 632 that assist in modulating the thermal environment surrounding a capacitance signal processing circuit 634.

FIG. 36 shows the capacitance sensor assembly 620 preferably passes signals between the signal processing circuit 204 and the capacitance signal processing circuit 634, as well as through a communication port 316, useful for transferring processed signals to a brainwave processing system (such as 334 of FIG. 24) for analysis.

In a preferred embodiment, a component chamber 636, provides a plurality of attachment tangs 638 used to secure the capacitance probe assembly 622 firmly positioned within the component chamber 636 of the capacitance sensor assembly 620, as shown by FIG. 36. In one embodiment of the capacitance sensor assembly 620, the capacitance probe assembly 622 is offset from the signal processing circuit 204 by a compressible member 640, and communicates with the signal processing circuit 204 via an electrical connection assembly 642 of FIG. 36.

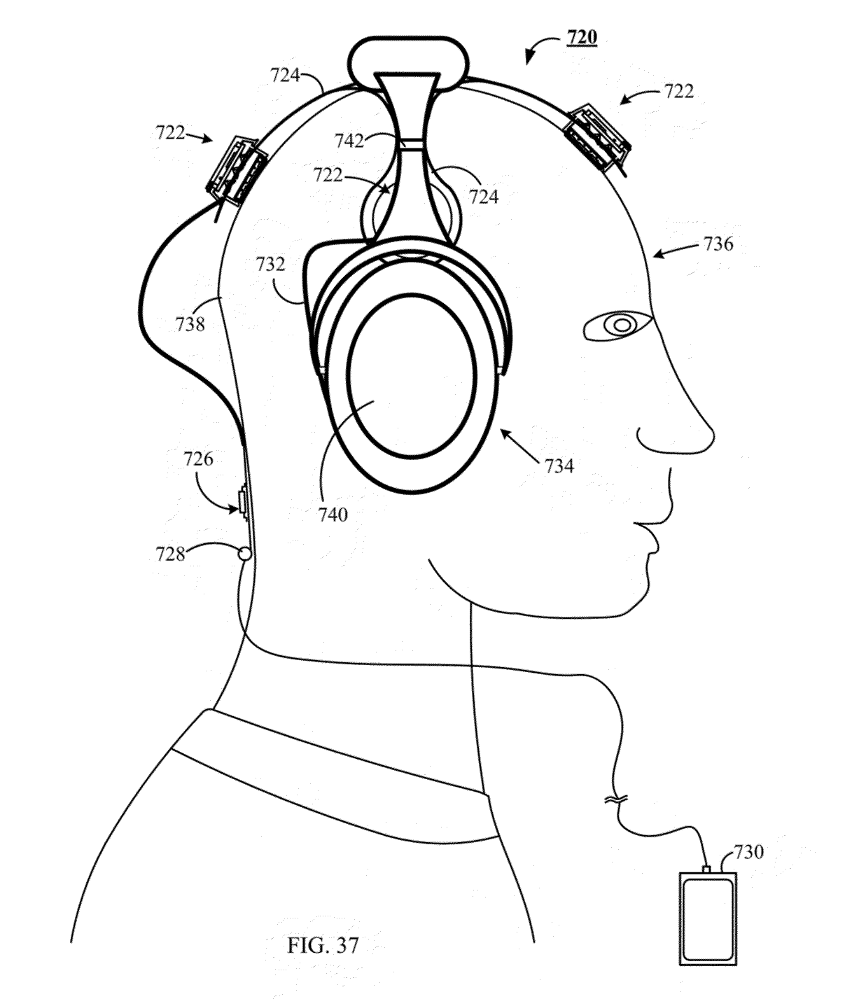

FIG. 37 shows a preferred configuration of an inventive standalone neurophysiologic performance measurement and training system 720, which preferably includes at least four sensor assemblies 722, (wherein 720 is selected from sensor assemblies 200, 300, 330, 600, or 620) supported by a sensor assembly retention web 724, a preferred brainwave processing system 726 that includes a multi-channel user interface 728 electrically interacting with an electronic device 730, which is preferably a portable computing and communication device, and a ground reference 732 interacting with an ear 734 of a subject 736 and electrically interacting with the preferred brain wave processing system 726. Preferably, the sensor web assembly is formed to support each of the sensor assemblies 722, provide a communication buss between the brainwave processing system 726 and each of the sensor assemblies 722 and the ground reference 732, and facilitate a pressing contact interface between each of the sensor assemblies 722 and a cranium 738 of the subject 736. Preferably, the sensor assemblies 722 may be of any type of neurophysiologic monitoring sensor including, but not limited to, the dry sensor assembly, such as 300, or the capacitance probe sensor such as 600 or 620.

FIG. 37 further shows the neurophysiologic performance measurement and training system 720 preferably further includes a head phone set 740, secured to the sensor assembly retention web 724 by an attachment member 742, which preferably is an attachment clip 742.

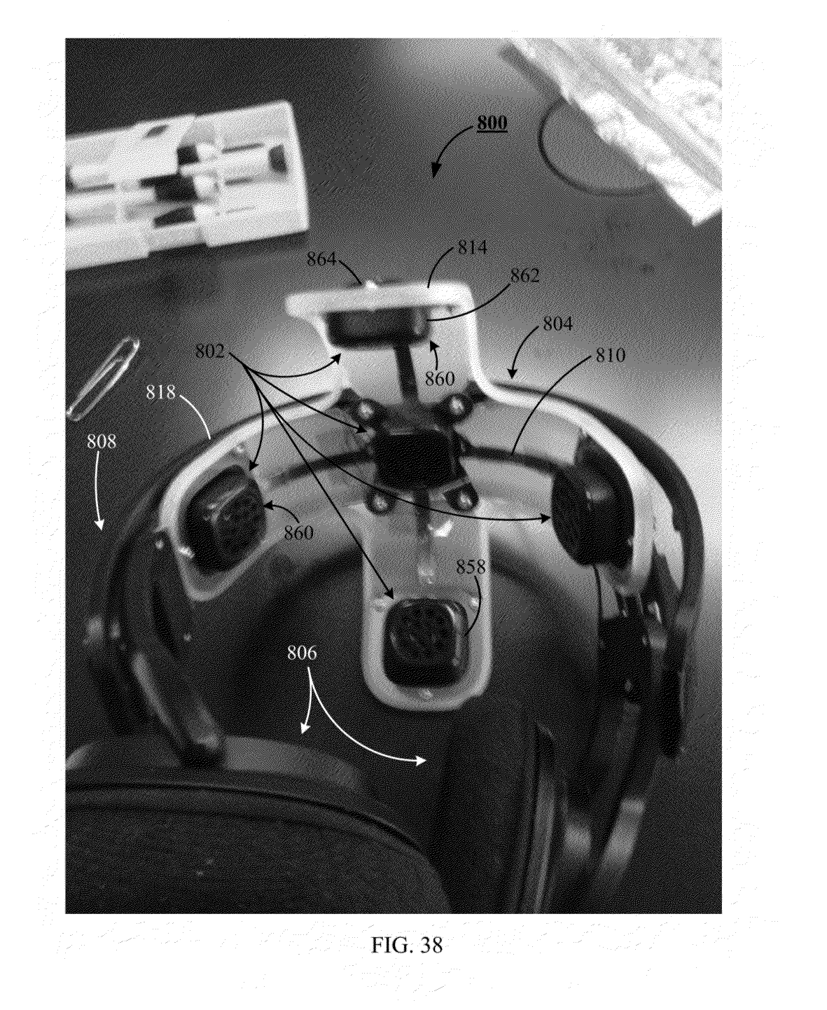

FIG. 38 shows an embodiment exemplary of a novel neurophysiological training headset 800 (“headset 800”), which includes a plurality of sensor assemblies 802 secured to a retention web 804. Each of the sensor assemblies 802 are configured to provide contact with the cranium 738 of the subject 736 (each of FIG. 37). The headset 800 preferably further provides headphones 806 (also referred to as earphones 806) secured to the retention web 804 by an attachment member 808, and frame assembly 810 communicating with each of the plurality of sensors 802.

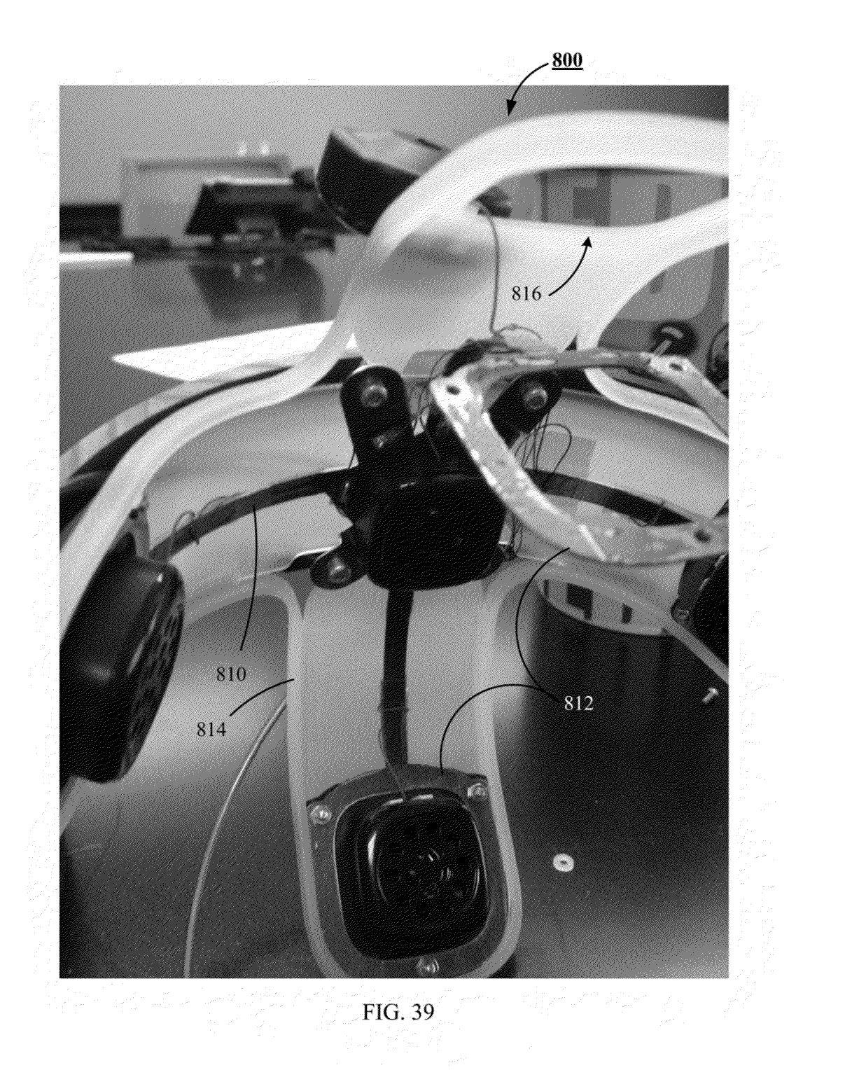

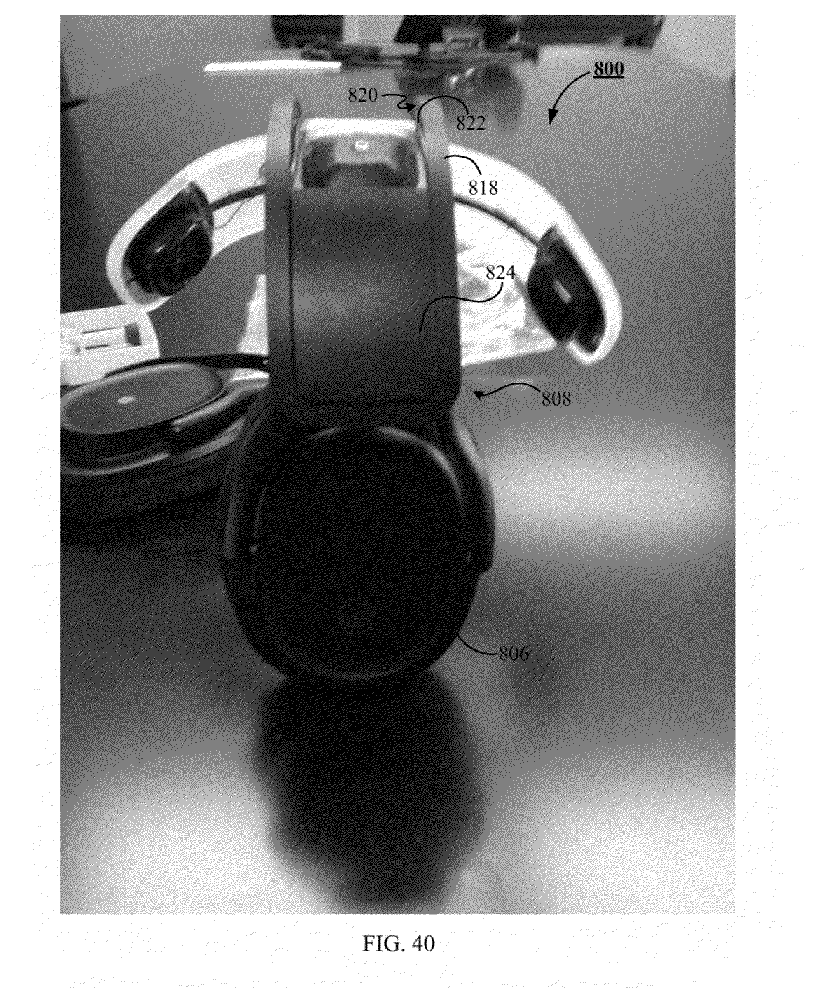

FIG. 39 shows the frame assembly 810 provides a sensor mounting plate 812 corresponding to each of the plurality of sensor assemblies 802, and a shape retention bracket 814 secured to the frame assembly 810. The shape retention bracket 814 providing a mounting aperture 816 for a preselected number of sensor assemblies of the plurality of sensor assemblies 802. Preferably, each of the preselected number of sensor assemblies 802 is disposed between the shape retention bracket 814 and their corresponding sensor mounting plates 812, while being confined within their corresponding mounting apertures 816.

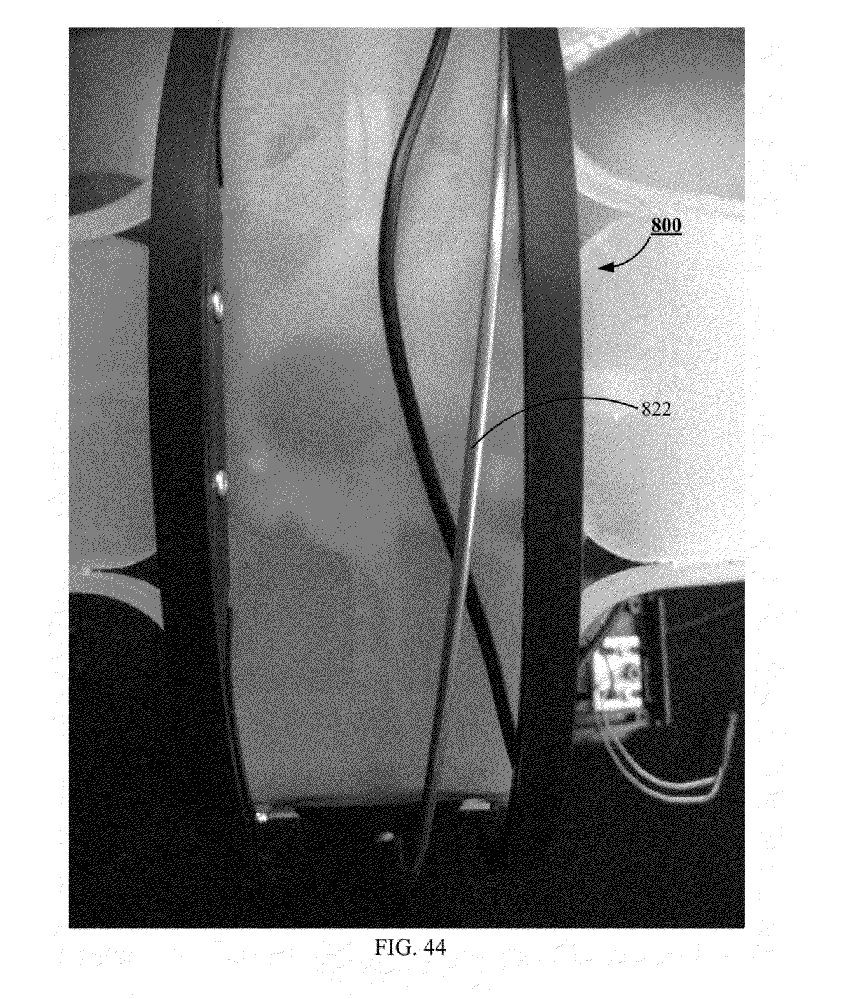

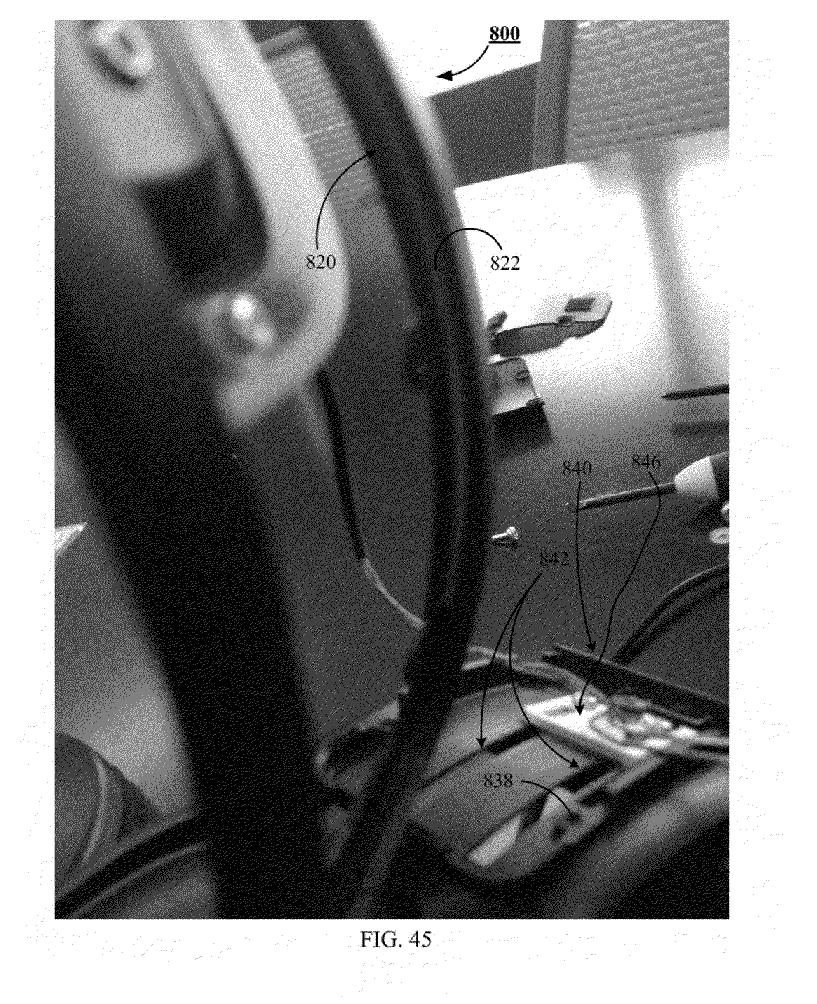

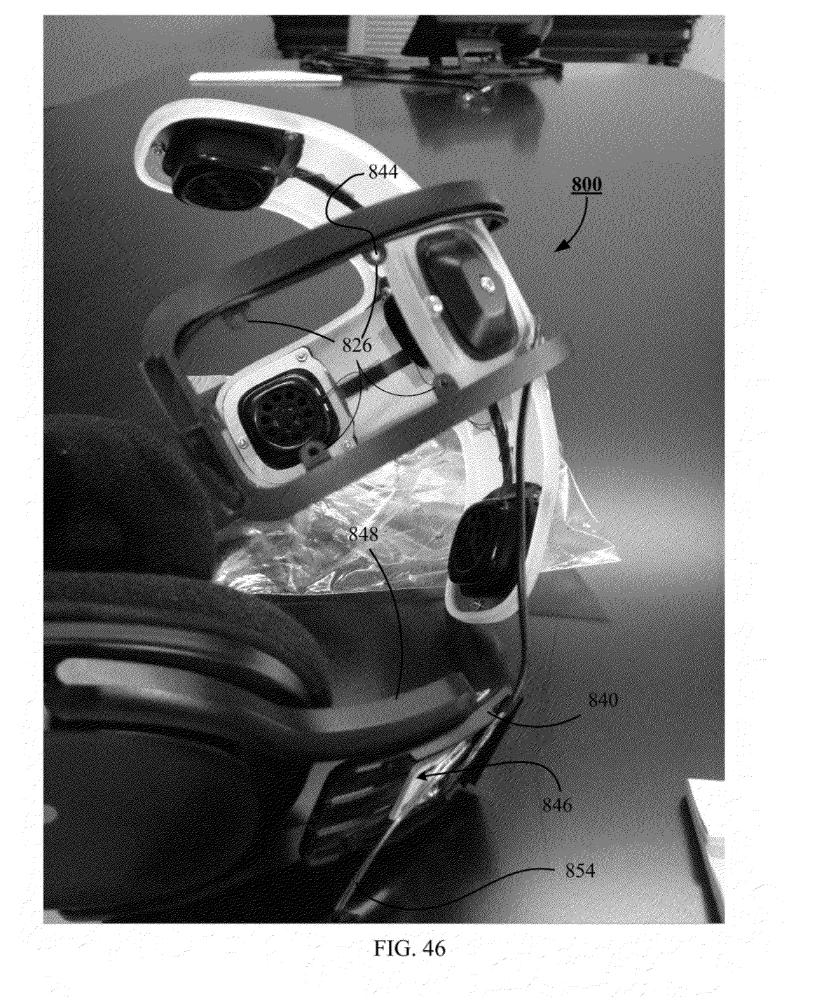

FIG. 40 shows the preferred attachment member 808 features a support structure 818 that provides a shape retention channel 820. The shape retention channel 820 cooperates with a shape retention member 822. FIG. 44 shows the shape retention member 822 in greater detail, while FIG. 45 shows the shape retention channel 820 in greater detail. FIG. 40 further shows an access cover 824, which provides access to a mounting flange 826 (shown by FIG. 46), of the attachment member 808. A back side view of the access cover 824 is shown by FIG. 43.

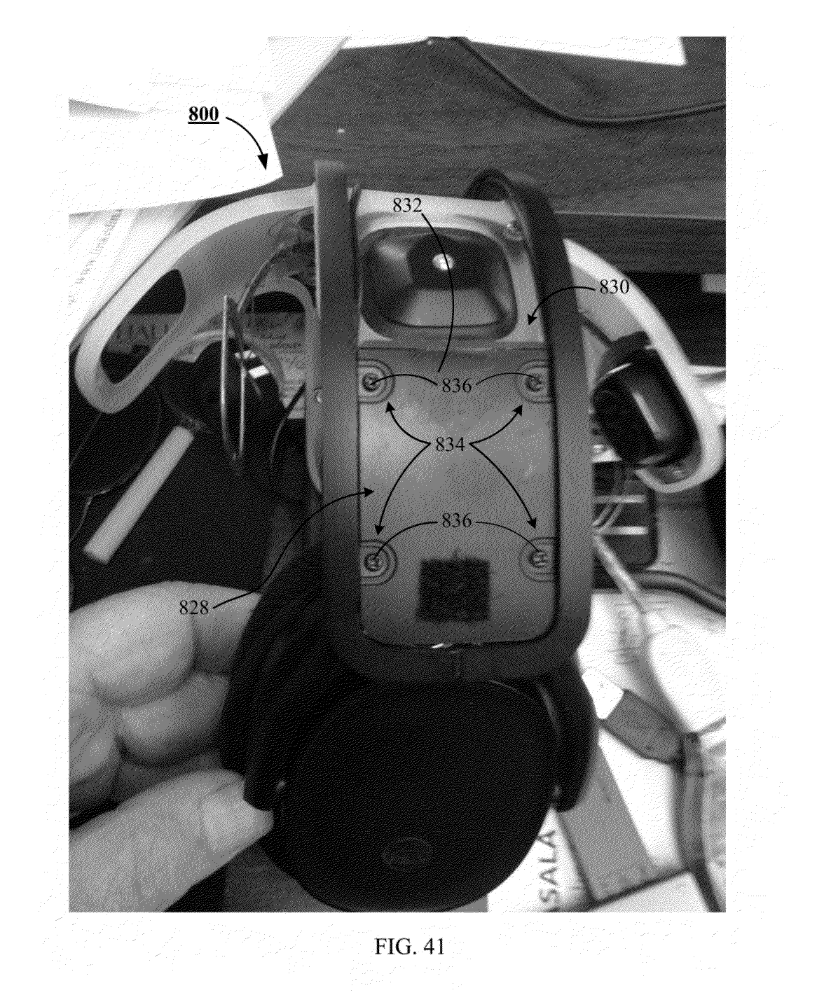

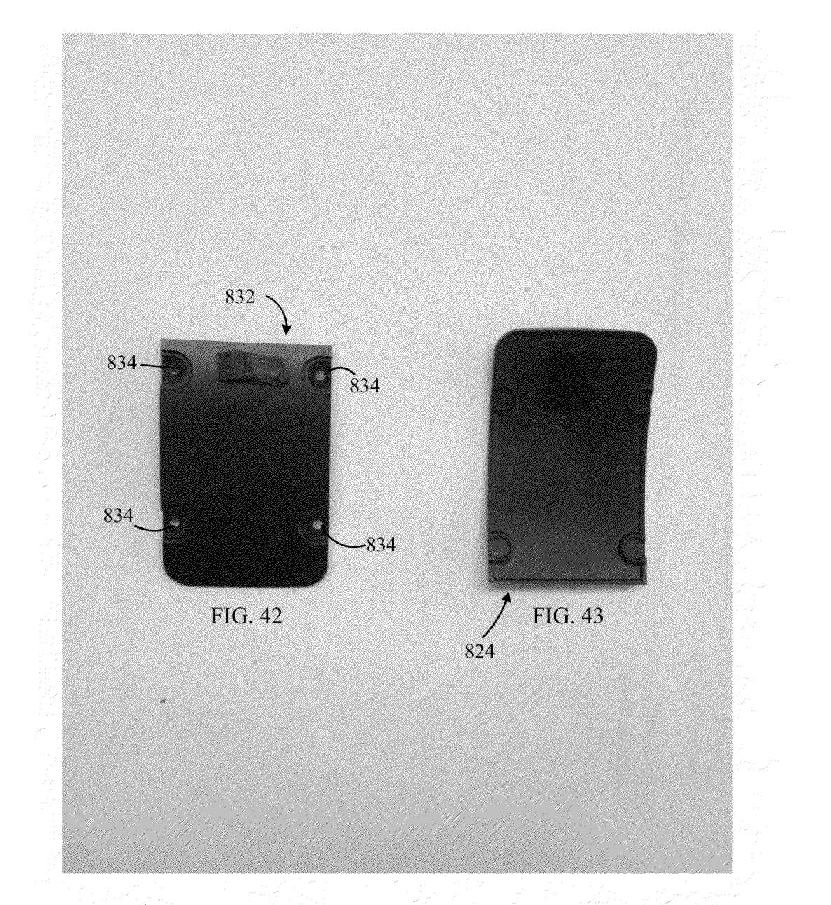

FIG. 41 reveals a continuously active tensioning mechanism 828, when the access cover 824 (of FIG. 40) is removed. Preferably, the continuously active tensioning mechanism 828 includes a tension housing 830 secured to the mounting flange 826, a cover plate 832 providing securement apertures 834, which are also shown by FIG. 42. The securement apertures 834 accommodate attachment structures 836, which communicate with corresponding mount apertures 838 of a guide plate 840 as shown by FIG. 45.

FIG. 45 further shows the guide plate 840 provides elongated guide apertures 842. Preferably, the securement aperture 834 of the cover plate 832 (each of FIG. 42) correspond to the mount apertures 838, and the mounting flange 826 (of FIG. 46) is disposed between the securement apertures 834 and the mount aperture 842. The mounting flange 826 provides access apertures 844 (of FIG. 46), corresponding to the mount apertures 838 and the securement aperture 834, which collectively facilitates use of the attachment structures 836 to secure the tension housing 830 (of FIG. 41) to the mounting flange 826 of FIG. 46.

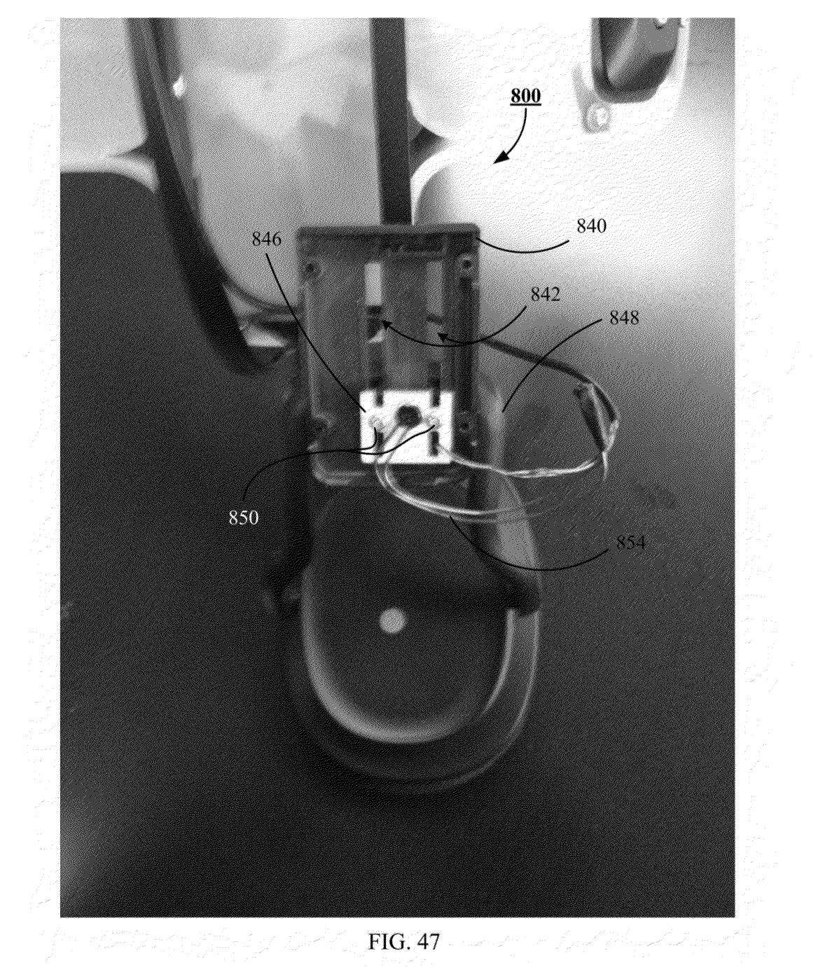

FIG. 46 shows a slide plate 846 cooperating with the guide plate 840; and an earphone mount 848 attached to the slide plate 846, while FIG. 47 shows linking hardware 850 connecting the slide plate 846 with said glide plate 840. Preferably, the linking hardware 850 protrudes through the elongated guide aperture 842 such that the guide plate 840 is in sliding contact with and disposed between each the slide plate 846 and the earphone mount 848.

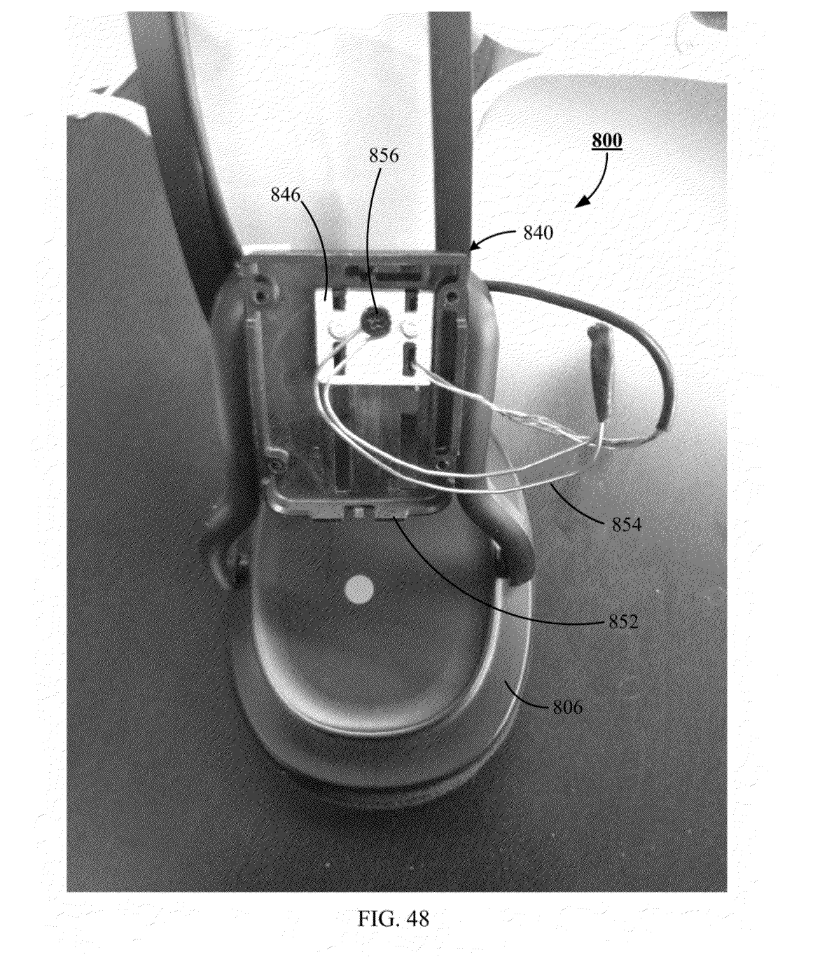

FIG. 48 shows the guide plate 840 provides a boss 852, which serves as a constraint for a tension member 854. In a preferred embodiment, a tension stay mount 856 communicates with the slide plate 846 to secure the tension member 854 in a fixed position relative to the slide plate 846. In a preferred embodiment, the tension member 854 interacting with the boss 852, of the guide plate 840, of the tension housing 830, such that when the earphone 806 is positioned in contact adjacency with the ear of the subject 736 (of FIG. 37), the tension member 854 acting on tension housing 830 promotes continuous force induced contact adjacency of the sensor assemblies 802, with the cranium 738 (of FIG. 37), of the subject 736.

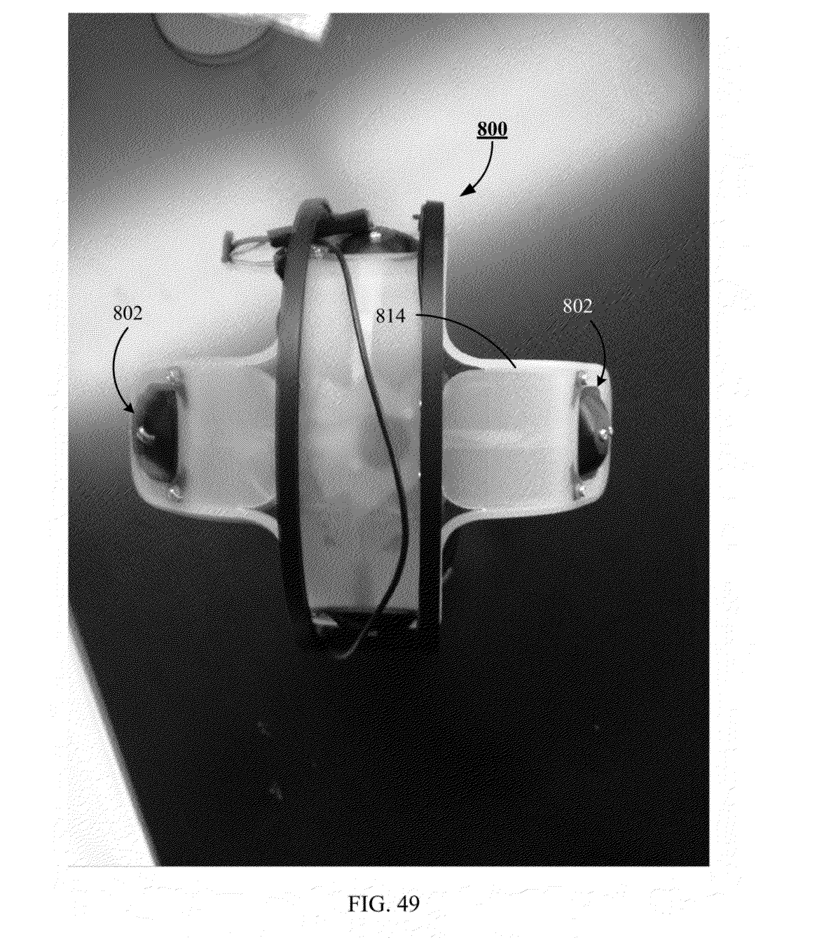

FIG. 49 is a top plan view of the neurophysiological training headset 800, which includes the plurality of sensor assemblies 802 secured to the retention web 804, by way of the shape retention bracket 814. Each of the sensor assemblies 802 are configured to provide contact with the cranium 738 of the subject 736 (each of FIG. 37).

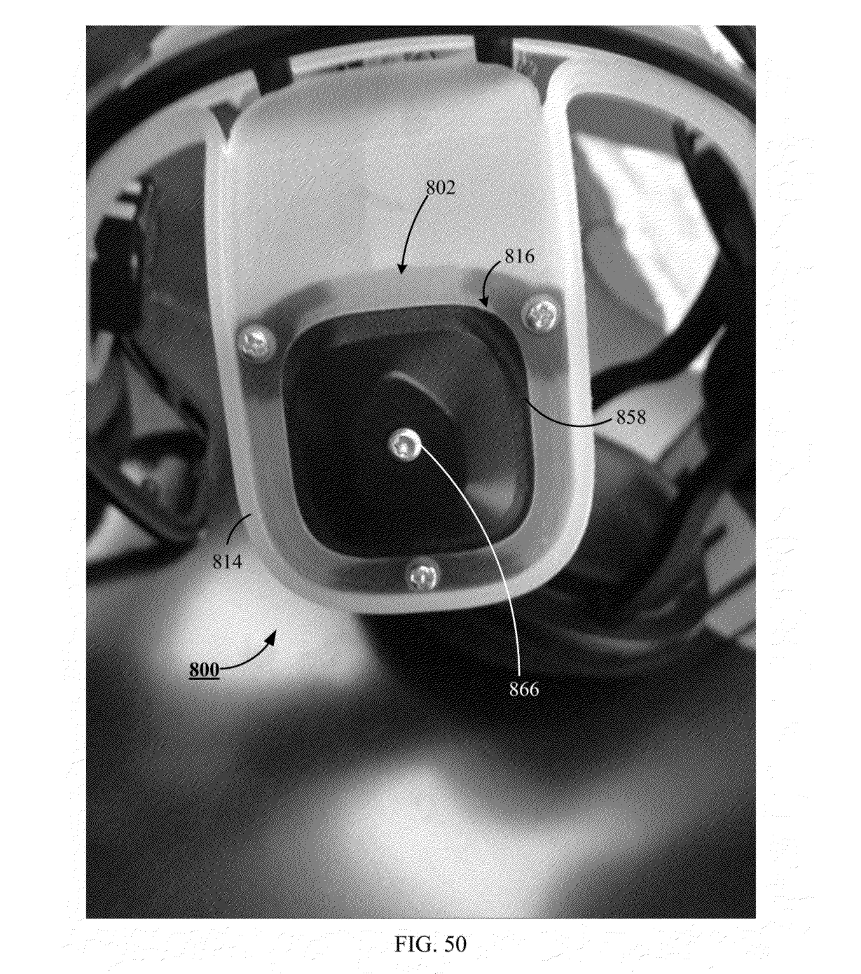

FIG. 50 shows a plan view of the sensor assembly 802, which includes at least a sensor housing 860 (of FIG. 38) that confines a sensor probe assembly, such as 10 of FIG. 1, and a signal processing circuit, such as 204 of FIG. 18. FIG. 50 further shows the shape retention bracket 814, provides the mounting aperture 816, which encloses or surrounds the sensor assembly. Additionally shown by FIG. 50, is a pliable compliant member 858 disposed within the mounting aperture 816, secured to the shape retention bracket 814, and attached to the sensor housing 860. In a preferred embodiment, the pliable compliant member 858 imparts a plurality of degrees of freedom of movement of the sensor assembly 802, the pliable compliant member 858 maintains conformance of the conductive pins in conductive contact with the cranium of the subject.

Returning to FIG. 38, additionally shown therein is a sensor housing 860 (housing 860) that preferably includes a main body 862, and a sensor securement cap 864 communicating with the main body 862, and in which the pliable compliant member 858 (also shown in FIG. 50), is further disposed between the main body 862 and the sensor securement cap 864. Preferably, a fastener 866 secures the securement cap 864 to the main body 862, and imparts a compressive load on the pliable compliant member 858 when the fastener 866 is fully engaged.

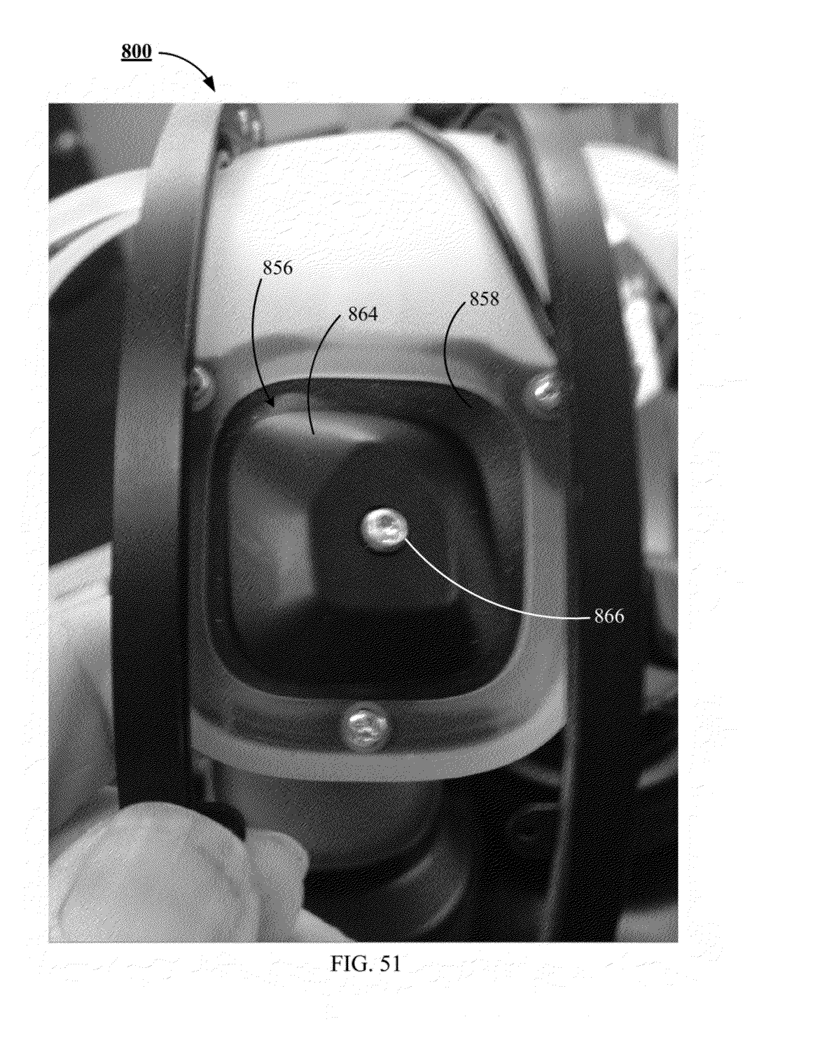

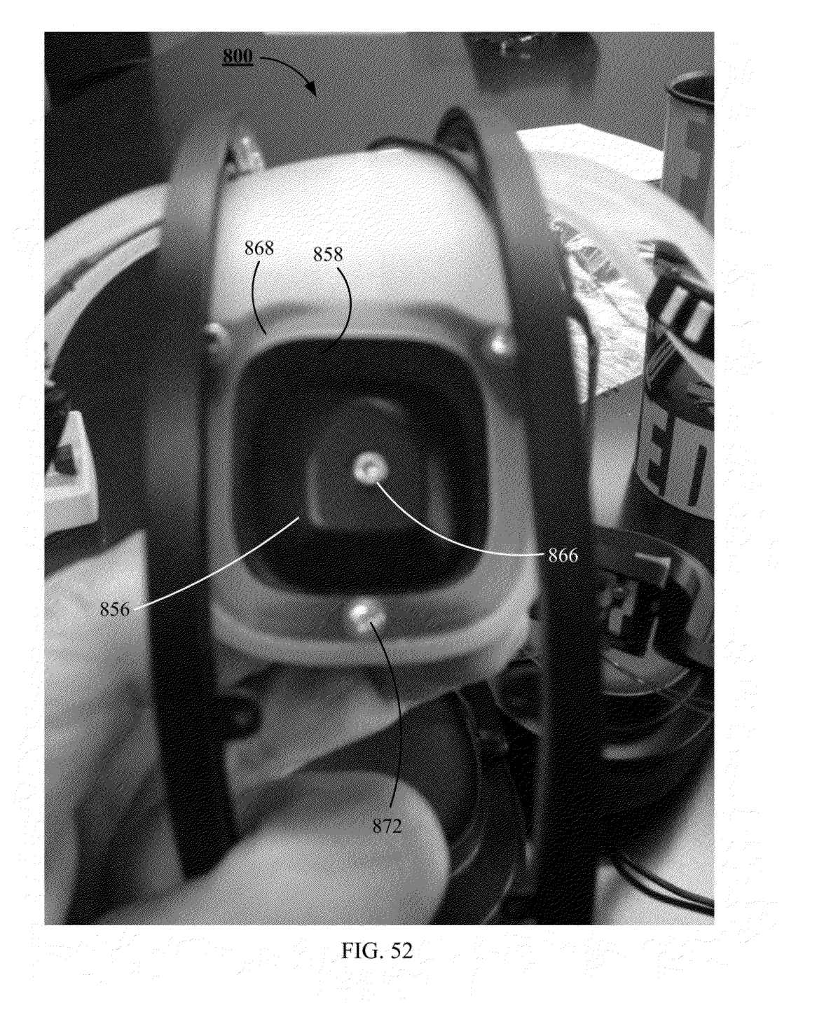

FIGS. 51 and 52 show the pliability provided by the pliable compliant member 858, enables the sensor assembly to move in the X-Y-Z axis, and well as roll, pitch, and yaw for an ability to provide a full six degrees of freedom of movement for the sensor assembly 802.

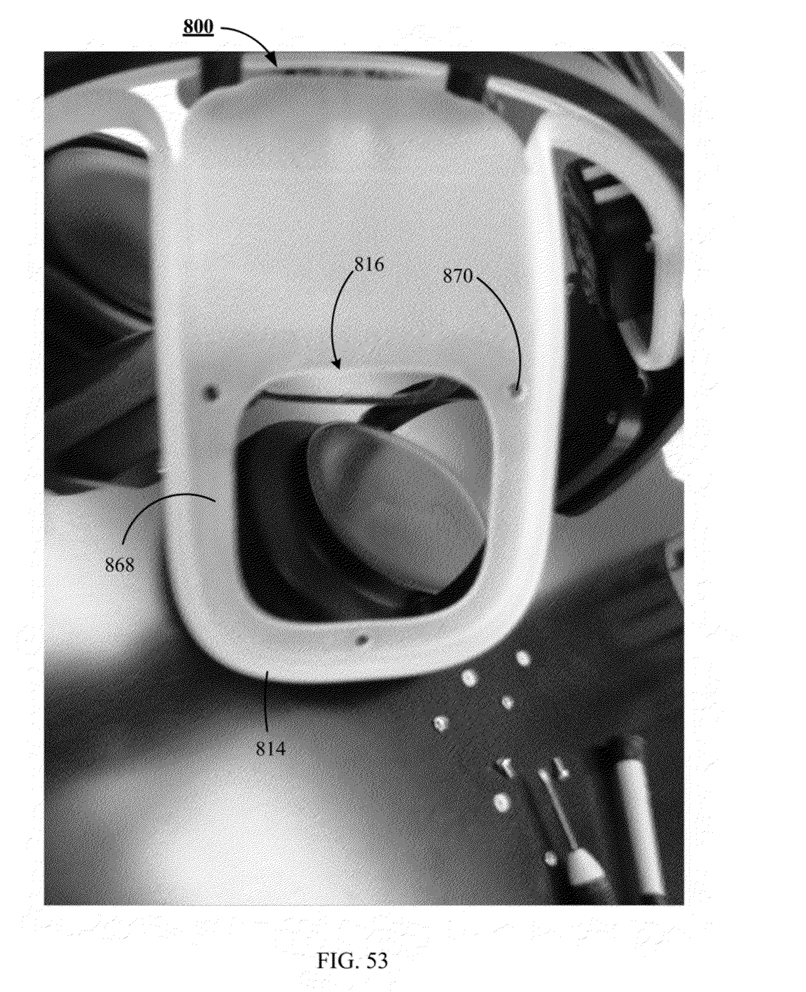

FIG. 53 shows the shape retention bracket 814 further provides a sensor mount flange 868 that includes at least one sensor fastening aperture 870. The sensor fastening aperture 870 facilitates passage of attachment structures 872 of FIG. 52. In a preferred embodiment, the pliable compliant member 856 of FIG. 52, is disposed between said sensor mounting plate 812, of FIG. 39, and the sensor mount flange 868. The mounting aperture 816, is enclosed by a sensor mount flange 868, provided by said shape retention bracket 814, and the sensor mounting plate 812 is provided by the frame assembly 810, of FIG. 39.

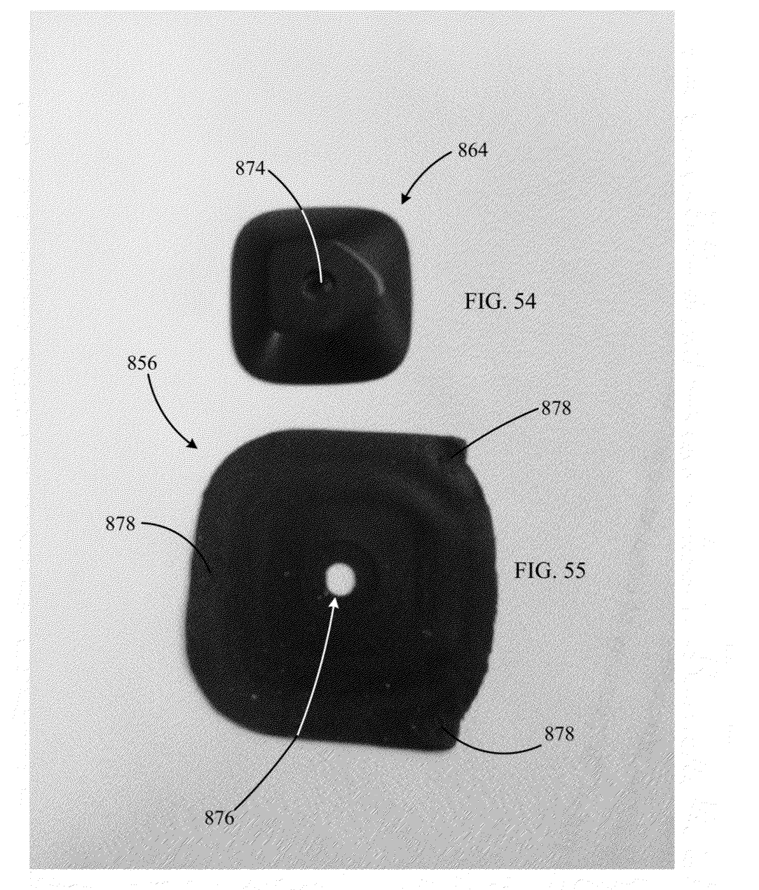

FIG. 54 shows the sensor securement cap 864 provides a fastener aperture 874, through which the fastener 866, of FIG. 38, secures the sensor securement cap 864 to the main body 862, and facilitates the compressive load to be imparted on the pliable compliant member 856 when the fastener 866 is fully engaged. While FIG. 55 shows that the pliable compliant member 856 provides a pass-through aperture 876, which accommodates passage of the fastener 866, of FIG. 52, such that the fastener 866 may communicate with the main body 862, of FIG. 38. FIG. 55 further shows that the pliable compliant member 858 additionally provides access apertures 878, which accommodates passage of the attachment structures 872, of FIG. 52.

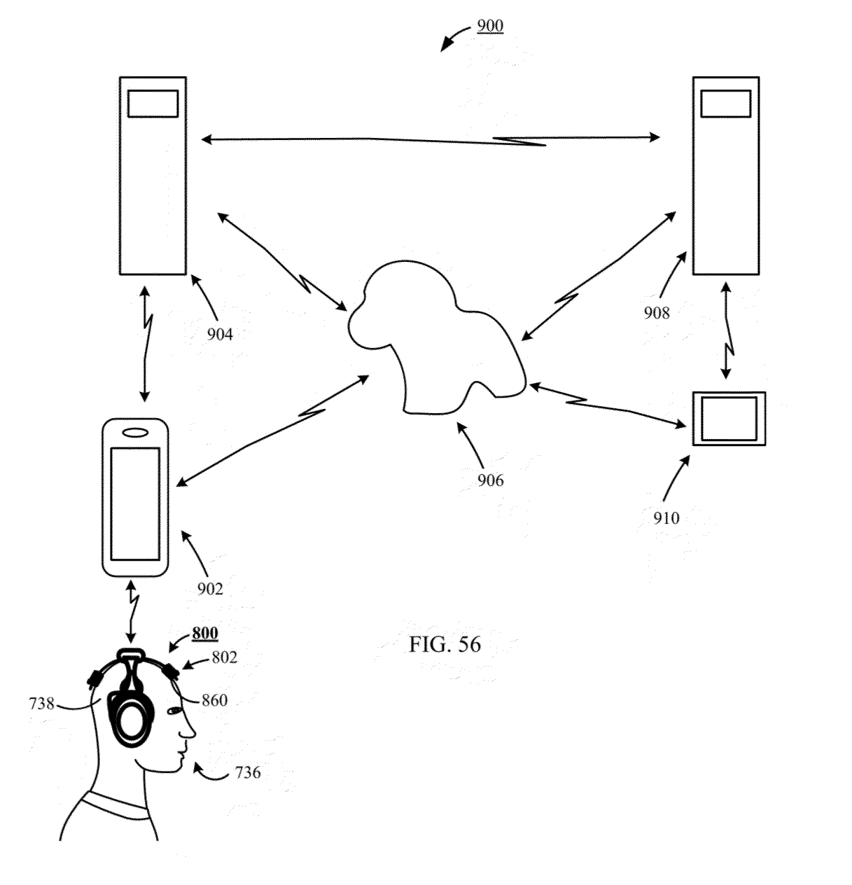

FIG. 56 shows a preferred embodiment of a neurophysiological training system 900, which preferably includes the neurophysiological training headset 800, affixed to the cranium 738 of the subject 736, and interacting with a communication device 902, which may communicate with a first edge router 904 either directly, or through a cloud 906. The first edge router 904 (also referred to as the first server 904) may communicate with a second edge router 908, either directly or via the cloud 906. The second edge router 908 (also referred to herein as the second server 908) preferably includes high performers data base and diagnostic software, which analyzes neurophysiological data (also referred to as brainwave data) of the subject collected by the neurophysiological training headset 800, and provides brain state status of the subject, based on an analysis of the collected neurophysiological data, to a computing device 910 for access by a brain training specialist.

In an operational mode, the neurophysiological training headset 800 interacts with the subject 736 and provides the sensor assembly 802. The sensor assembly 802 collects brainwave data of said subject 736. The communication device 904, cooperating with said neurophysiological training headset 800, the communication device 902 transmits the collected brainwave data to the second server 908, via either the first server 904, or the cloud 906, interacting with the communication device. FIG. 56 further shows a computing device 910, linked with the second server 908, the computing device 910 analyzes the collected brainwave data, determines a brain training regimen based on the collected brainwave data and a high performance brainwave data base resident in the second server 908. The computing device 910, downloads the determined brain training regimen to the communication device 902, monitors the subject's performance in executing said training regimen, adjusts the training regimen based on the monitored performance, and downloads the adjusted training regimen for use by the subject 736. In an alternate embodiment, the communication device is located within the housing 860 of the sensor assembly 802.

As will be apparent to those skilled in the art, a number of modifications could be made to the preferred embodiments which would not depart from the spirit or the scope of the present invention. While the presently preferred embodiments have been described for purposes of this disclosure, numerous changes and modifications will be apparent to those skilled in the art. Insofar as these changes and modifications are within the purview of the appended claims, they are to be considered as part of the present invention.

1. A device comprising:
a plurality of sensor assemblies;
a retention web securing each of the plurality of sensor assemblies, said sensor assemblies adjacent a predetermined location about a cranium of a subject; and
a headphone secured to said retention web by an attachment member, said attachment member provides a continuously active tensioning mechanism, said continuously active tensioning mechanism promotes continuous force induced contact adjacency of said sensor assemblies with said cranium of the subject.

2. The device of claim 1, in which said retention web comprising:
a frame assembly, said frame assembly providing a sensor mounting plate for each sensor assembly of the plurality of sensor assemblies; and
a shape retention bracket secured to the frame assembly, said shape retention bracket providing a mounting aperture for a preselected number of sensor assemblies of the plurality of sensor assemblies, wherein each of said preselected number of sensor assemblies is disposed between said corresponding shape retention bracket and said corresponding sensor mounting plate, and confined within said mounting aperture.

3. The device of claim 1, in which said attachment member further comprising:
a support structure, said support structure providing a shape retention channel;
a shape retention member cooperating with the shape retention channel;
a mounting flange adjacent the shape retention channel; and
an access cover enclosing the mounting flange.

4. The device of claim 3, in which said continuously active tensioning mechanism comprising:
a tension housing secured to said mounting flange; and
a slide structure interacting with the tension housing.

5. The device of claim 4, in which said tension housing comprising:
a guide plate providing an elongated guide aperture and a mount aperture;
a cover plate providing a securement aperture corresponding to said mount aperture, and wherein said mounting flange is disposed between said securement aperture and said mount aperture, said mounting flange providing an access aperture corresponding to said mount aperture and said securement aperture; and
an attachment structure securing said securement aperture to said mount aperture by way of said access aperture.

6. The device of claim 5, in which said slide structure comprising:
a slide plate cooperating with said guide plate;
an earphone mount attached to the slide plate; and
linking hardware connecting said slide plate with said glide plate, said linking hardware protruding through said elongated guide aperture such that said guide plate is in sliding contact with and disposed between each the slide plate and the earphone mount.

7. The device of claim 6, in which said slide structure further comprising:
a tension stay mounted to said slide plate, and
a tension member secured to said tension stay, said tension member interacting with said tension housing, such that when said earphone is positioned in contact adjacency with a car of said subject, said tension member acting on said tension housing promotes continuous force induced contact adjacency of said sensor assemblies with said cranium of the subject.

8. The device of claim 2, in which said attachment member further comprising:
a support structure, said support structure providing a shape retention channel;
a shape retention member cooperating with the shape retention channel;
a mounting flange adjacent the shape retention channel; and
an access cover enclosing the mounting flange.

9. The device of claim 8, in which said continuously active tensioning mechanism comprising:
a tension housing secured to said mounting flange; and
a slide structure interacting with the tension housing.

10. The device of claim 9, in which said tension housing comprising:
a guide plate providing an elongated guide aperture and a mount aperture;
a cover plate providing a securement aperture corresponding to said mount aperture, and wherein said mounting flange is disposed between said securement aperture and said mount aperture, said mounting flange providing an access aperture corresponding to said mount aperture and said securement aperture; and
an attachment structure securing said securement aperture to said mount aperture by way of said access aperture.

11. The device of claim 10, in which said slide structure comprising:
a slide plate cooperating with said guide plate;
an earphone mount attached to the slide plate; and
linking hardware connecting said slide plate with said glide plate, said linking hardware protruding through said elongated guide aperture such that said guide plate is in sliding contact with and disposed between each the slide plate and the earphone mount.

12. The device of claim 11, in which said slide structure further comprising:
a tension stay mounted to said slide plate; and
a tension member secured to said tension stay, said tension member interacting with said tension housing, such that when said earphone is positioned in contact adjacency with a ear of said subject, said tension member acting on said tension housing promotes continuous force induced contact adjacency of said sensor assemblies with said cranium of the subject.
